SEQUENCE LISTING (156 sequences)

SEQ ID NO: 1 - protein, 9 aa, synthetic construct
  REGION 1..9: note = Breast cancer targeting peptide
  REGION 3..4: note = MISC_FEATURE - Xaa can be any amino acid other than C, R, W or Y
  REGION 6..8: note = MISC_FEATURE - Xaa can be any amino acid other than C, R, W or Y
CRXXRXXXC

SEQ ID NO: 2 - protein, 11 aa, synthetic construct
  REGION 1..11: note = Breast cancer targeting peptide
  SITE 3: note = MISC_FEATURE - Xaa can be D or E
  SITE 7: note = MISC_FEATURE - Xaa can be Q or N
RGXPAYXGRFL

SEQ ID NO: 3 - protein, 7 aa, synthetic construct
  REGION 1..7: note = Breast cancer targeting peptide
  SITE 2: note = MISC_FEATURE - Xaa is any amino acid, except for C, W and Y
PXLNVSP

SEQ ID NO: 4 - protein, 6 aa, synthetic construct
  REGION 1..6: note = Pancreatic cancer targeting peptide
  SITE 2: note = MISC_FEATURE - Xaa can be any amino acid, except C, Y and W
  SITE 4: note = MISC_FEATURE - Xaa can be any amino acid, except C, Y and W
PXIXIT

SEQ ID NO: 5 - protein, 9 aa, synthetic construct
  REGION 1..9: note = Lung cancer targeting peptide
  SITE 1: note = MISC_FEATURE - Xaa is any amino acid except for W, Y and C
  SITE 4: note = MISC_FEATURE - Xaa is any amino acid except for W, Y and C
  REGION 6..7: note = MISC_FEATURE - Xaa is any amino acid except for W, Y and C
  SITE 9: note = MISC_FEATURE - Xaa is any amino acid except for W, Y and C
XPWXEXXYX

SEQ ID NO: 6 - protein, 9 aa, synthetic construct
  REGION 1..9: note = Breast cancer targeting peptide
CSNIDARAC

SEQ ID NO: 7 - protein, 9 aa, synthetic construct
  REGION 1..9: note = Breast cancer targeting peptide
CDPSRGKNC

SEQ ID NO: 8 - protein, 9 aa, synthetic construct
  REGION 1..9: note = Breast cancer targeting peptide
CPSDLKDAC

SEQ ID NO: 9 - protein, 9 aa, synthetic construct
  REGION 1..9: note = Breast cancer targeting peptide
CRTTRGTKC

SEQ ID NO: 10 - protein, 9 aa, synthetic construct
  REGION 1..9: note = Breast cancer targeting peptide
CRMTRNKPC

SEQ ID NO: 11 - protein, 9 aa, synthetic construct
  REGION 1..9: note = Breast cancer targeting peptide
CRVSRQNKC

SEQ ID NO: 12 - protein, 9 aa, synthetic construct
  REGION 1..9: note = Breast cancer targeting peptide
CAKIDPELC

SEQ ID NO: 13 - protein, 9 aa, synthetic construct
  REGION 1..9: note = Breast cancer targeting peptide
CGGERGKSC

SEQ ID NO: 14 - protein, 8 aa, synthetic construct
  REGION 1..8: note = Breast cancer targeting peptide
KGVSLSYR

SEQ ID NO: 15 - protein, 6 aa, synthetic construct
  REGION 1..6: note = Breast cancer targeting peptide
RTRYED

SEQ ID NO: 16 - protein, 6 aa, synthetic construct
  REGION 1..6: note = Breast cancer targeting peptide
GMMYRS

SEQ ID NO: 17 - protein, 6 aa, synthetic construct
  REGION 1..6: note = Breast cancer targeting peptide
RWRTNF

SEQ ID NO: 18 - protein, 6 aa, synthetic construct
  REGION 1..6: note = Breast cancer targeting peptide
RIPLEM

SEQ ID NO: 19 - protein, 6 aa, synthetic construct
  REGION 1..6: note = Breast cancer targeting peptide
QFDEPR

SEQ ID NO: 20 - protein, 6 aa, synthetic construct
  REGION 1..6: note = Breast cancer targeting peptide
TSAVRT

SEQ ID NO: 21 - protein, 6 aa, synthetic construct
  REGION 1..6: note = Breast cancer targeting peptide
GLWQGP

SEQ ID NO: 22 - protein, 6 aa, synthetic construct
  REGION 1..6: note = Breast cancer targeting peptide
QCTGRF

SEQ ID NO: 23 - protein, 6 aa, synthetic construct
  REGION 1..6: note = Breast cancer targeting peptide
LPGMMG

SEQ ID NO: 24 - protein, 6 aa, synthetic construct
  REGION 1..6: note = Breast cancer targeting peptide
DVGTTE

SEQ ID NO: 25 - protein, 6 aa, synthetic construct
  REGION 1..6: note = Breast cancer targeting peptide
TDLGAM

SEQ ID NO: 26 - protein, 6 aa, synthetic construct
  REGION 1..6: note = Breast cancer targeting peptide
DSNAES

SEQ ID NO: 27 - protein, 6 aa, synthetic construct
  REGION 1..6: note = Breast cancer targeting peptide
ITDMAA

SEQ ID NO: 28 - protein, 6 aa, synthetic construct
  REGION 1..6: note = Breast cancer targeting peptide
WRPCES

SEQ ID NO: 29 - protein, 6 aa, synthetic construct
  REGION 1..6: note = Breast cancer targeting peptide
WRNTIA

SEQ ID NO: 30 - protein, 6 aa, synthetic construct
  REGION 1..6: note = Breast cancer targeting peptide
IDKQLE

SEQ ID NO: 31 - protein, 6 aa, synthetic construct
  REGION 1..6: note = Breast cancer targeting peptide
FMEIET

SEQ ID NO: 32 - protein, 6 aa, synthetic construct
  REGION 1..6: note = Breast cancer targeting peptide
HEVVAG

SEQ ID NO: 33 - protein, 6 aa, synthetic construct
  REGION 1..6: note = Breast cancer targeting peptide
GGHTRQ

SEQ ID NO: 34 - protein, 6 aa, synthetic construct
  REGION 1..6: note = Breast cancer targeting peptide
INGKVT

SEQ ID NO: 35 - protein, 12 aa, synthetic construct
  REGION 1..12: note = Breast cancer targeting peptide
  SITE 4: note = misc_feature - Xaa can be any naturally occurring amino acid
VPWXEPAYQRFL

SEQ ID NO: 36 - protein, 4 aa, synthetic construct
  REGION 1..4: note = Breast cancer targeting peptide
GRDS

SEQ ID NO: 37 - protein, 10 aa, synthetic construct
  REGION 1..10: note = Breast cancer targeting peptide
RGEPAYQRFL

SEQ ID NO: 38 - protein, 10 aa, synthetic construct
  REGION 1..10: note = Breast cancer targeting peptide
RGDPAYQRFL

SEQ ID NO: 39 - protein, 11 aa, synthetic construct
  REGION 1..11: note = Breast cancer targeting peptide
  SITE 2: note = misc_feature - Xaa can be any naturally occurring amino acid
WXEPAYQGRFL

SEQ ID NO: 40 - protein, 11 aa, synthetic construct
  REGION 1..11: note = Breast cancer targeting peptide
  SITE 2: note = misc_feature - Xaa can be any naturally occurring amino acid
WXEPAYNGRFL

SEQ ID NO: 41 - protein, 11 aa, synthetic construct
  REGION 1..11: note = Breast cancer targeting peptide
RGEPAYQGRFL

SEQ ID NO: 42 - protein, 11 aa, synthetic construct
  REGION 1..11: note = Breast cancer targeting peptide
RGDPAYQGRFL

SEQ ID NO: 43 - protein, 11 aa, synthetic construct
  REGION 1..11: note = Breast cancer targeting peptide
RGEPAYNGRFL

SEQ ID NO: 44 - protein, 11 aa, synthetic construct
  REGION 1..11: note = Breast cancer targeting peptide
RGDPAYNGRFL

SEQ ID NO: 45 - protein, 10 aa, synthetic construct
  REGION 1..10: note = Breast cancer targeting peptide
  SITE 2: note = misc_feature - Xaa can be any naturally occurring amino acid
WXEPAYQRFL

SEQ ID NO: 46 - protein, 10 aa, synthetic construct
  REGION 1..10: note = Breast cancer targeting peptide
  SITE 2: note = misc_feature - Xaa can be any naturally occurring amino acid
AXEPAYQRFL

SEQ ID NO: 47 - protein, 10 aa, synthetic construct
  REGION 1..10: note = Breast cancer targeting peptide
WAEPAYQRFL

SEQ ID NO: 48 - protein, 10 aa, synthetic construct
  REGION 1..10: note = Breast cancer targeting peptide
  SITE 2: note = misc_feature - Xaa can be any naturally occurring amino acid
WXAPAYQRFL

SEQ ID NO: 49 - protein, 10 aa, synthetic construct
  REGION 1..10: note = Breast cancer targeting peptide
  SITE 2: note = misc_feature - Xaa can be any naturally occurring amino acid
WXEAAYQRFL

SEQ ID NO: 50 - protein, 10 aa, synthetic construct
  REGION 1..10: note = Breast cancer targeting peptide
  SITE 2: note = misc_feature - Xaa can be any naturally occurring amino acid
WXEPAAQRFL

SEQ ID NO: 51 - protein, 10 aa, synthetic construct
  REGION 1..10: note = Breast cancer targeting peptide
  SITE 2: note = misc_feature - Xaa can be any naturally occurring amino acid
WXEPAYARFL

SEQ ID NO: 52 - protein, 10 aa, synthetic construct
  REGION 1..10: note = Breast cancer targeting peptide
  SITE 2: note = misc_feature - Xaa can be any naturally occurring amino acid
WXEPAYQAFL

SEQ ID NO: 53 - protein, 10 aa, synthetic construct
  REGION 1..10: note = Breast cancer targeting peptide
  SITE 2: note = misc_feature - Xaa can be any naturally occurring amino acid
WXEPAYQAAL

SEQ ID NO: 54 - protein, 10 aa, synthetic construct
  REGION 1..10: note = Breast cancer targeting peptide
  SITE 2: note = misc_feature - Xaa can be any naturally occurring amino acid
WXEPAYQAFA

SEQ ID NO: 55 - protein, 10 aa, synthetic construct
  REGION 1..10: note = Breast cancer targeting peptide
  SITE 2: note = misc_feature - Xaa can be any naturally occurring amino acid
EXEPAYQRFL

SEQ ID NO: 56 - protein, 10 aa, synthetic construct
  REGION 1..10: note = Breast cancer targeting peptide
  SITE 2: note = misc_feature - Xaa can be any naturally occurring amino acid
LXEPAYQRFL

SEQ ID NO: 57 - protein, 10 aa, synthetic construct
  REGION 1..10: note = Breast cancer targeting peptide
  SITE 2: note = misc_feature - Xaa can be any naturally occurring amino acid
KXEPAYQRFL

SEQ ID NO: 58 - protein, 15 aa, synthetic construct
  REGION 1..15: note = Melanoma targeting peptide
PRPGAPLAGSWPGTS

SEQ ID NO: 59 - protein, 12 aa, synthetic construct
  REGION 1..12: note = Melanoma targeting peptide
ADGAPRPGAPLA

SEQ ID NO: 60 - protein, 15 aa, synthetic construct
  REGION 1..15: note = Melanoma targeting peptide
DRWRPALPVVLFPLH

SEQ ID NO: 61 - protein, 15 aa, synthetic construct
  REGION 1..15: note = Melanoma targeting peptide
ASSSYPLIHWRPWAR

SEQ ID NO: 62 - protein, 8 aa, synthetic construct
  REGION 1..8: note = Melanoma targeting peptide#
DRWRPALP

SEQ ID NO: 63 - protein, 8 aa, synthetic construct
  REGION 1..8: note = Melanoma targeting peptide
IHWRPWAR

SEQ ID NO: 64 - protein, 15 aa, synthetic construct
  REGION 1..15: note = Melanoma targeting peptide
AAEWLDAFFVRHVDR

SEQ ID NO: 65 - protein, 15 aa, synthetic construct
  REGION 1..15: note = Melanoma targeting peptide
GDVWLFLTSTSHFAR

SEQ ID NO: 66 - protein, 14 aa, synthetic construct
  REGION 1..14: note = Melanoma targeting peptide
GCSVSSVGALCTHV

SEQ ID NO: 67 - protein, 15 aa, synthetic construct
  REGION 1..15: note = Melanoma targeting peptide
APCCSHLDASPFQRP

SEQ ID NO: 68 - protein, 14 aa, synthetic construct
  REGION 1..14: note = Melanoma targeting peptide
AQSNFVTWGYNVAV

SEQ ID NO: 69 - protein, 15 aa, synthetic construct
  REGION 1..15: note = Melanoma targeting peptide
RASDVGSDVVPRYPF

SEQ ID NO: 70 - protein, 24 aa, synthetic construct
  REGION 1..24: note = Melanoma targeting peptide
CRGDCGGKWCFRVCYRGICYRRCR

SEQ ID NO: 71 - protein, 10 aa, synthetic construct
  REGION 1..10: note = Melanoma targeting peptide
TAASGVRSMH

SEQ ID NO: 72 - protein, 10 aa, synthetic construct
  REGION 1..10: note = Melanoma targeting peptide
LTLRWVGLMS

SEQ ID NO: 73 - protein, 9 aa, synthetic construct
  REGION 1..9: note = Melanoma targeting peptide
CVLNGRMEC

SEQ ID NO: 74 - protein, 11 aa, synthetic construct
  REGION 1..11: note = Melanoma targeting peptide
KKEKDIMKKTI

SEQ ID NO: 75 - protein, 7 aa, synthetic construct
  REGION 1..7: note = Melanoma targeting peptide
GRGDSPK

SEQ ID NO: 76 - protein, 7 aa, synthetic construct
  REGION 1..7: note = Melanoma targeting peptide
CRGDGWC

SEQ ID NO: 77 - protein, 9 aa, synthetic construct
  REGION 1..9: note = Prostate cancer targeting peptide
CGRRAGGSC

SEQ ID NO: 78 - protein, 24 aa, synthetic construct
  REGION 1..24: note = Prostate cancer targeting peptide
CRGDCGGKWCFRVCYRGICYRRCR

SEQ ID NO: 79 - protein, 7 aa, synthetic construct
  REGION 1..7: note = Prostate cancer targeting peptide
RPARPAR

SEQ ID NO: 80 - protein, 8 aa, synthetic construct
  REGION 1..8: note = Prostate cancer targeting peptide
CRGDKGPD

SEQ ID NO: 81 - protein, 9 aa, synthetic construct
  REGION 1..9: note = Prostate cancer targeting peptide
CRGDRGPDC

SEQ ID NO: 82 - protein, 9 aa, synthetic construct
  REGION 1..9: note = Prostate cancer targeting peptide
CRGDKTTNC

SEQ ID NO: 83 - protein, 9 aa, synthetic construct
  REGION 1..9: note = Prostate cancer targeting peptide
CRGDHAGDC

SEQ ID NO: 84 - protein, 9 aa, synthetic construct
  REGION 1..9: note = Prostate cancer targeting peptide
CRGDHGVEC

SEQ ID NO: 85 - protein, 9 aa, synthetic construct
  REGION 1..9: note = Prostate cancer targeting peptide
CGRGDNLPC

SEQ ID NO: 86 - protein, 9 aa, synthetic construct
  REGION 1..9: note = Prostate cancer targeting peptide
CGRGDNLAC

SEQ ID NO: 87 - protein, 9 aa, synthetic construct
  REGION 1..9: note = Prostate cancer targeting peptide
CEKRGDNLC

SEQ ID NO: 88 - protein, 6 aa, synthetic construct
  REGION 1..6: note = Sarcoma targeting peptide
NGRAHA

SEQ ID NO: 89 - protein, 31 aa, synthetic construct
  REGION 1..31: note = Sarcoma targeting peptide
KDEPQRRSARLSAKPAPPKPEPKPKKAPAKK

SEQ ID NO: 90 - protein, 5 aa, synthetic construct
  REGION 1..5: note = Sarcoma targeting peptide
CREKA

SEQ ID NO: 91 - protein, 9 aa, synthetic construct
  REGION 1..9: note = Sarcoma targeting peptide
TDCTPSRCT

SEQ ID NO: 92 - protein, 9 aa, synthetic construct
  REGION 1..9: note = Sarcoma targeting peptide
SWCQFEKCL

SEQ ID NO: 93 - protein, 9 aa, synthetic construct
  REGION 1..9: note = Sarcoma targeting peptide
VPCRFKQCW

SEQ ID NO: 94 - protein, 9 aa, synthetic construct
  REGION 1..9: note = Sarcoma targeting peptide
CTAMRNTDC

SEQ ID NO: 95 - protein, 8 aa, synthetic construct
  REGION 1..8: note = Sarcoma targeting peptide
CRESLKNC

SEQ ID NO: 96 - protein, 8 aa, synthetic construct
  REGION 1..8: note = Sarcoma targeting peptide
CMEMGVKC

SEQ ID NO: 97 - protein, 12 aa, synthetic construct
  REGION 1..12: note = Lung cancer targeting peptide
SYDILKPNPQRL

SEQ ID NO: 98 - protein, 12 aa, synthetic construct
  REGION 1..12: note = Lung cancer targeting peptide
SHGKPPSFSPYT

SEQ ID NO: 99 - protein, 9 aa, synthetic construct
  REGION 1..9: note = Lung cancer targeting peptide
CLSYYPSYC

SEQ ID NO: 100 - protein, 6 aa, synthetic construct
  REGION 1..6: note = Lung cancer targeting peptide
RLQLKL

SEQ ID NO: 101 - protein, 8 aa, synthetic construct
  REGION 1..8: note = Lung cancer targeting peptide
GHGKHKNK

SEQ ID NO: 102 - protein, 12 aa, synthetic construct
  REGION 1..12: note = Lung cancer targeting peptide
TDSILRSYDWTY

SEQ ID NO: 103 - protein, 12 aa, synthetic construct
  REGION 1..12: note = Lung cancer targeting peptide
DMPKQLLAPWYY

SEQ ID NO: 104 - protein, 12 aa, synthetic construct
  REGION 1..12: note = Lung cancer targeting peptide
SYPLSFLGPLIS

SEQ ID NO: 105 - protein, 9 aa, synthetic construct
  REGION 1..9: note = Colorectal cancer targeting peptide
CPKSNNGVC

SEQ ID NO: 106 - protein, 9 aa, synthetic construct
  REGION 1..9: note = Colorectal cancer targeting peptide
CKTPNGHLC

SEQ ID NO: 107 - protein, 9 aa, synthetic construct
  REGION 1..9: note = Colorectal cancer targeting peptide
CQSISTAHC

SEQ ID NO: 108 - protein, 9 aa, synthetic construct
  REGION 1..9: note = Colorectal cancer targeting peptide
CNDDVPNKC

SEQ ID NO: 109 - protein, 9 aa, synthetic construct
  REGION 1..9: note = Colorectal cancer targeting peptide
CEIPGKVVC

SEQ ID NO: 110 - protein, 9 aa, synthetic construct
  REGION 1..9: note = Colorectal cancer targeting peptide
CLRTPANHC

SEQ ID NO: 111 - protein, 10 aa, synthetic construct
  REGION 1..10: note = SSTR targeting peptide
DFCFDWKTCT

SEQ ID NO: 112 - protein, 10 aa, synthetic construct
  REGION 1..10: note = SSTR targeting peptide
DFCYDWKVCW

SEQ ID NO: 113 - protein, 14 aa, synthetic construct
  REGION 1..14: note = GRPR targeting peptide
YQRLGNQWAVGHLM

SEQ ID NO: 114 - protein, 5 aa, synthetic construct
  REGION 1..5: note = apoptotic cascade targeting peptide
DWLPK

SEQ ID NO: 115 - protein, 21 aa, synthetic construct
  REGION 1..21: note = CXCR4 targeting peptide
LGASWHRPDKCCLGYQKRPLP

SEQ ID NO: 116 - protein, 10 aa, synthetic construct
  REGION 1..10: note = CXCR4 targeting peptide
NAACAWLEAQ

SEQ ID NO: 117 - protein, 7 aa, synthetic construct
  REGION 1..7: note = CXCR4 targeting peptide
RACRFFC

SEQ ID NO: 118 - protein, 35 aa, synthetic construct
  REGION 1..35: note = Cell membrane targeting peptide
ACEQNPIYWARYADWLFTTPLLLLDLALLVDADET

SEQ ID NO: 119 - protein, 42 aa, synthetic construct
  REGION 1..42: note = Temperature regulating targeting peptide
VSSLESKVSSLESKVSKLESKKSKLESKVSKLESKVSSLESK

SEQ ID NO: 120 - protein, 5 aa, synthetic construct
  REGION 1..5: note = Temperature regulating targeting peptide
  SITE 4: note = misc_feature - Xaa can be any naturally occurring amino acid
VPGXG

SEQ ID NO: 121 - protein, 9 aa, synthetic construct
  REGION 1..9: note = MC1R targeting peptide
LDHDFRWGL

SEQ ID NO: 122 - protein, 8 aa, synthetic construct
  REGION 1..8: note = Granyzme B targeting peptide
AGGIEFAD

SEQ ID NO: 123 - protein, 6 aa, synthetic construct
  REGION 1..6: note = xVb3 targeting peptide
RGDDYK

SEQ ID NO: 124 - protein, 33 aa, synthetic construct
  REGION 1..33: note = Integrins targeting peptide
GCPRPRGDNPPLTCSQDSDCLAGCVCGPNGFCG

SEQ ID NO: 125 - protein, 5 aa, synthetic construct
  REGION 1..5: note = CD13 targeting peptide
CNGRC

SEQ ID NO: 126 - protein, 18 aa, synthetic construct
  REGION 1..18: note = Integrins targeting peptide
LRRFSTMPFMFNINNVNF

SEQ ID NO: 127 - protein, 10 aa, synthetic construct
  REGION 1..10: note = MMP2/9 targeting peptide
CTTHWGFTLC

SEQ ID NO: 128 - protein, 21 aa, synthetic construct
  REGION 1..21: note = VEGFR2 targeting peptide
CVNHPAFACHTMYYHHYQHHL

SEQ ID NO: 129 - protein, 9 aa, synthetic construct
  REGION 1..9: note = p32 targeting peptide
CGNKRTRGC

SEQ ID NO: 130 - protein, 9 aa, synthetic construct
  REGION 1..9: note = Lymphatic targeting peptide
CAGRRSAYC

SEQ ID NO: 131 - protein, 9 aa, synthetic construct
  REGION 1..9: note = Lymphatic targeting peptide
CREAGRKAC

SEQ ID NO: 132 - protein, 9 aa, synthetic construct
  REGION 1..9: note = Tumor lymphatic targeting peptide
CLSDGKRKC

SEQ ID NO: 133 - protein, 9 aa, synthetic construct
  REGION 1..9: note = NRP-1 targeting peptide
CRGDKGPDC

SEQ ID NO: 134 - protein, 16 aa, synthetic construct
  REGION 1..16: note = Cell cytoplasm targeting peptide
RQIKIWFQNRRMKWKK

SEQ ID NO: 135 - protein, 12 aa, synthetic construct
  REGION 1..12: note = M2/TAM targeting peptide
YEQDPWGVKWWY

SEQ ID NO: 136 - protein, 9 aa, synthetic construct
  REGION 1..9: note = MDGI targeting peptide
CGLSGLGVA

SEQ ID NO: 137 - protein, 10 aa, synthetic construct
  REGION 1..10: note = Fibrosis targeting peptide
CGLIIQKNEC

SEQ ID NO: 138 - protein, 9 aa, synthetic construct
  REGION 1..9: note = IL13RA2 targeting peptide
CGEMGWVRC

SEQ ID NO: 139 - protein, 19 aa, synthetic construct
  REGION 1..19: note = LRP-1 targeting peptide
TFFYGGSRGKRNNFKTEEY

SEQ ID NO: 140 - protein, 19 aa, synthetic construct
  REGION 1..19: note = Angiopep targeting peptide
TFFYGGSRGRRNNFRTEEY

SEQ ID NO: 141 - protein, 12 aa, synthetic construct
  REGION 1..12: note = Tenascin-c targeting peptide
FHKHKSPALSPV

SEQ ID NO: 142 - protein, 7 aa, synthetic construct
  REGION 1..7: note = NRP-1 targeting peptide
CGNKRTR

SEQ ID NO: 143 - protein, 4 aa, synthetic construct
  REGION 1..4: note = Integrins targeting peptide
RGDV

SEQ ID NO: 144 - protein, 8 aa, synthetic construct
  REGION 1..8: note = Melanoma targeting peptide
GPSRVGGC

SEQ ID NO: 145 - protein, 9 aa, synthetic construct
  REGION 1..9: note = Cancer targeting peptide
GPSRVGGCD

SEQ ID NO: 146 - protein, 9 aa, synthetic construct
  REGION 1..9: note = Cancer targeting peptide
  SITE 9: note = MISC_FEATURE - Xaa is a gamma carboxy-glutamic acid
GPSRVGGCX

SEQ ID NO: 147 - protein, 7 aa, synthetic construct
  REGION 1..7: note = Cancer targeting peptide
GPSRVGG

SEQ ID NO: 148 - protein, 11 aa, synthetic construct
  REGION 1..11: note = Breast cancer targeting peptide
  SITE 2: note = MISC_FEATURE - Xaa can be any amino acid, except for W and C
  SITE 11: note = MISC_FEATURE - Xaa is gamma-carboxyglutamic acid
WXAPAYQRFLX

SEQ ID NO: 149 - protein, 9 aa, synthetic construct
  REGION 1..9: note = Breast cancer targeting peptide
  SITE 2: note = MISC_FEATURE - Xaa is a hydrophobic amino acid, such as I, L, A or V
  SITE 3: note = MISC_FEATURE - Xaa can be any amino acid except C and W
  SITE 4: note = MISC_FEATURE - Xaa is a hydrophobic amino acid, such as I, L, A or V
  SITE 7: note = MISC_FEATURE - Xaa can be any amino acid except C and W
SXXXSWXPP

SEQ ID NO: 150 - protein, 9 aa, synthetic construct
  REGION 1..9: note = Breast cancer targeting peptide
  SITE 2: note = MISC_FEATURE - Xaa is a hydrophobic amino acid, such as I, L, A or V
  SITE 3: note = MISC_FEATURE - Xaa can be any amino acid, except for C and W
  SITE 4: note = MISC_FEATURE - Xaa is a hydrophobic amino acid, such as I, L, A or V
  SITE 7: note = MISC_FEATURE - Xaa can be any amino acid, except for C and W
TXXXTWXPP

SEQ ID NO: 151 - protein, 9 aa, synthetic construct
  REGION 1..9: note = Breast cancer targeting peptide
  SITE 2: note = MISC_FEATURE - Xaa is a a hydrophobic amino acid, such as I, L, A or V
  SITE 3: note = MISC_FEATURE - Xaa can be any amino acid, except for C and W
  SITE 4: note = MISC_FEATURE - Xaa is a a hydrophobic amino acid, such as I, L, A or V
  SITE 7: note = MISC_FEATURE - Xaa can be any amino acid, except for C and W
SXXXTWXPP

SEQ ID NO: 152 - protein, 9 aa, synthetic construct
  REGION 1..9: note = Breast cancer targeting peptide
  SITE 2: note = MISC_FEATURE - Xaa is a hydrophobic amino acid, such as I, L, A or V
  SITE 3: note = MISC_FEATURE - Xaa can be any amino acid, except for C and W
  SITE 4: note = MISC_FEATURE - Xaa is a hydrophobic amino acid, such as I, L, A or V
  SITE 7: note = MISC_FEATURE - Xaa can be any amino acid, except for C and W
TXXXSWXPP

SEQ ID NO: 153 - protein, 8 aa, synthetic construct
  REGION 1..8: note = Glioblastoma targeting peptide
  REGION 7..8: note = MISC_FEATURE - Xaa can be any amino acid except for C, W and Y
VDLPEHXX

SEQ ID NO: 154 - protein, 8 aa, synthetic construct
  REGION 1..8: note = Glioblastoma targeting peptide
  REGION 7..8: note = MISC_FEATURE - Xaa can be any amino acid except for C, W and Y
VDLPTHXX

SEQ ID NO: 155 - protein, 8 aa, synthetic construct
  REGION 1..8: note = Glioblastoma targeting peptide
  REGION 7..8: note = MISC_FEATURE - Xaa can be any amino acid except for C, W and Y
VGLPEHXX

SEQ ID NO: 156 - protein, 8 aa, synthetic construct
  REGION 1..8: note = Glioblastoma targeting peptide
  REGION 7..8: note = MISC_FEATURE - Xaa can be any amino acid except for C, W and Y
VGLPTHXX

(19) United States
(12) Patent Application Publication
(10) Pub. No.: US 2024/0190922 A1
(43) Pub. Date: Jun. 13, 2024

(54) PEPTIDE PLATINUM COMPLEXES AND METHODS OF USE THEREOF

(71) Applicant: Oncovolution, LLC, Danvers, MA (US)

(72) Inventors: Mario DiPaola, Scituate, MA (US); Krzysztof Dziewiszek, Littleton, MA (US)

(21) Appl. No.: 18/048,293

(22) Filed: Oct. 20, 2022

Related U.S. Application Data
(63) Continuation of application No. PCT/US21/28613, filed on Apr. 22, 2021.
(60) Provisional application No. 63/013,832, filed on Apr. 22, 2020.

(51) Int. Cl. C07K 14/00; A61K 31/506; A61K 31/513; A61K 45/06; A61K 47/26; A61P 35/00

(52) U.S. Cl. CPC C07K 14/001; A61K 31/506; A61K 31/513; A61K 45/06; A61K 47/26; A61P 35/00

(57) ABSTRACT

This invention provides novel peptide platinum complexes, pharmaceutical compositions comprising a peptide platinum complex, and methods for treating cancer using a peptide platinum complex. Kits comprising a unit dosage form of a compound or composition of the invention are also provided.

### CROSS REFERENCE

This application is a continuation application of International Patent Application No. PCT/US2021/028613, filed on Apr. 22, 2021 which claims priority to U.S. Application No. 63/013,832 filed on Apr. 22, 2020, the content of each of which is herein incorporated by reference in its entirety.

### SEQUENCE LISTING

This application contains a Sequence Listing which has been submitted electronically as WIPO Standard ST.26 XML file, and is hereby incorporated by reference in its entirety. Said xml file, created on Oct. 20, 2022 is named 120506-10103_sequence_ST26.xml and is 55,168 bytes in size.

### FIELD OF THE INVENTION

The present invention relates to targeted delivery of platinum-based cancer agents for treatment of cancer.

### BACKGROUND OF THE INVENTION

Platinum coordination complexes were identified as cytotoxic agents in 1965. Cis-diamminedichloroplatinum (II) (cisplatin) is a clinically significant anticancer agent useful for the treatment of a broad spectrum of neoplastic diseases in humans (Loehrer et al., Ann. Int. Med. 100:704-713 (1984)). However, long-term treatment with cisplatin is limited by systemic toxicity, including emesis, nephrotoxicity, ototoxicity and neurotoxicity (Zwelling et al., “Platinum Complexes” in Pharmacologic Principles of Cancer Treatment, Ed. B. A. Chabner, Saunders, Philadelphia, PA (1982)). To improve the therapeutic index of cisplatin, numerous platinum analogs have been prepared and tested, typically with modest results (M. C. Christian, “The Current Status of Platinum Analogs”, Seminars in Oncology, 1992, 19(6), 720-733).

cis-Diammine(1,1-cyclobutanedicarboxylato)platinum (II) (carboplatin), is a second-generation platinum analog. Carboplatin is effective when used in place of cisplatin in established chemotherapeutic drug regimens and although less emetic, nephrotoxic, neurotoxic, and ototoxic than cisplatin, carboplatin has undesirable myelosuppressive properties that cisplatin does not (Go et al., J. Clin. Oncol. 1999, 17(1): 409-22).

Oxaliplatin is a more recently developed third-generation cisplatin analog with a 1,2-diaminocyclohexane (DACH) carrier ligand which has displayed clinical activity in a variety of tumor types and is not cross-resistant with cisplatin and carboplatin. Oxaliplatin acts synergistically with 5-fluorouracil (5-fluorouracil) in both 5-fluorouracil resistant and chemotherapy-naive disease and has also been evaluated as a single-agent and in combination regiments for treatment of breast, lung and prostate cancer and non-Hodgkin's lymphoma (Misset et al., Crit Rev. Oncol. Hematol. 2000, 35(2): 75-93).

Other platinum analogs have shown promise in clinical trials; these include NDDP (cis-bis-neodecanoato-trans-R,R-1,2-dicyclohexane platinum (II) (U.S. Pat. No. 5,178,876); nedaplatin (Latorre et al., Int. J. Oncol. 2002, 21(1):179-86); JM335 (trans-amine-(cyclohexylaminedichlorodihydroxo) platinum(IV)) (Kelland et al. J. Inorg. Biochem. 1999, 77(1-2):115-115); iproplatin (Martin, Clin. Breast Cancer 2001, 2(3):190-208); the dinuclear platinum complexes BBR3005 (trans-PtCl(NH₃)₂2H₂N(CH₂)₆NH₂)²⁺ and BBR3171 (cis-PtCl(NH₃)₂2H₂N(CH₂)₆NH₂)²⁺, and the trinuclear platinum complex BBR3464 (trans-PtCl(NH₃)2-2 mu-trans-Pt(NH₃)2(H₂N(CH₂)₆NH₂)₂)⁴⁺ (Roberts et al. J. Inorg. Biochem. 1999, 77(1-2):47-50); and the sterically hindered platinum complex, AMD473 (cis-aminedichloro(2-methylpyridine) platinum (II)) (Holford, et al. J. Cancer 1998, 77(3):366-73).

Despite the availability of many anticancer agents, traditional chemotherapy has several drawbacks (Stockdale, 1998, “Principles of Cancer Patient Management” in Scientific American Medicine, vol. 3, Rubenstein and Federman, eds., ch. 12, sect. 10)). Almost all anticancer agents are toxic, and chemotherapy can cause significant, and often dangerous side effects, including severe nausea, bone marrow depletion, immunosuppression, etc. Additionally, many tumor cells are resistant or can develop resistance to anticancer agents through multi-drug resistance. Therefore, there is a significant need in the art for novel agents with improved therapeutic indices that are useful for treating cancer and related proliferative diseases.

### SUMMARY OF THE DISCLOSURE

The invention provides peptide platinum complexes and their use to treat cancer. Peptide platinum complexes according to the invention, when administered to a subject, target delivery of the cytotoxic platinum to cancer cells. The platinum moiety of the complex confers cytotoxicity to cancer cells intercalating into the cancer cell DNA and causing cell death. The peptide(s) moiety confers specificity on the complex by directing the peptide platinum complex to cancer cells which contain a binding partner for the peptide. The binding partner may be a receptor for the peptide or another peptide-recognizing entity.

Accordingly, in one aspect, the present invention provides a purified complex of formula (I) (a “peptide platinum complex”) as below:

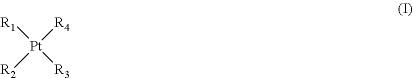

or pharmaceutically acceptable salts thereof,
   wherein
R₁ and R₂ are independently —N(R₆)₂, —NH₃⁺, or R₁ and R₂ are each —NH₂ and join through an C₂-C₆ alkylene or C₃-C₇ cycloalkylene group to form a bidentate diamine ligand;
R₃ is a peptide coordination ligand;
R₄ is a peptide coordination ligand, an inorganic coordination ligand, —Cl, —CN, —NO₃, —OH, H₂O or —OC(O)R₅;
R₅ is C₁-C₂₄ alkyl; and
each R₆ is independently —H, —C₁-C₆ alkyl, —C₃-C₇ cycloalkyl or -aryl.

Some embodiments include a purified peptide platinum complex having the formula (I):

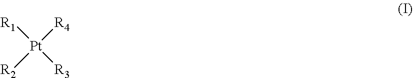

or a pharmaceutically acceptable salt thereof,
wherein
R₁ and R₂ are independently —N(R₆)₂, —NH₃⁺, or R₁ and R₂ are each —NH₂ and join through an C₂ -C₆ alkylene or C₃-C₇ cycloalkylene group to form a bidentate diamine ligand;
R₃ is a peptide ligand, with the proviso that R₃ cannot be an amino acid;
R₄ is a peptide ligand, an inorganic ligand, —CN or —OC(O)R₅, with the proviso that R₄ cannot be an amino acid;
R₅ is C₁-C₂₄ alkyl; and each R₆ is independently —H, —C₁-C₆ alkyl, —C₃-C₇ cycloalkyl or -aryl

Some embodiments include a purified chemotherapeutic complex comprises diaminocyclohexylcarbonato-platinate (II) (11). The chemotherapeutic complex comprises diaminocyclohexylcarbonato-platinate (II) (11) encapsulated into liposomes or encapsulated into biocompatible polymeric nanoparticles, such as polylacticglycolic acid (PLGA).

Any one of the peptide platinum complex as above where R3 and R4 are each, independently, a peptide. Any one of the peptide platinum complex as above wherein R3 and R4 are joined to form a bidentate peptide ligand. Any one of the peptide platinum complex as above wherein the peptide contains cysteine. Any one of the peptide platinum complex as above wherein the peptide contains methionine. Any one of the peptide platinum complex as above wherein the peptide terminal primary amino group is acylated. Any one of the peptide platinum complex as above wherein the peptide contains one or more of L-glycine, L-proline, L-serine, L-arginine, L-valine, and L-cysteine Any one of the peptide platinum complex as above wherein the peptide forms a complex with platinum via a linker. Any one of the peptide platinum complex as above wherein R₁ and R₂ are joined to form a bidentate diamine ligand. Any one of the peptide platinum complex as above wherein the bidentate diamine ligand is selected from the group consisting of trans-R,R-1,2-diaminocyclohexane, trans-S,S-1,2-diaminocyclohexane, cis-1,2-diaminocyclohexane or 1,2-ethylenediamine. Any one of the peptide platinum complex as above wherein R4 is an inorganic ligand, —CN or —OC(O)R5; wherein R5 is C1-C24 alkyl. Any one of the peptide platinum complex as above wherein R4 is Cl−, Br−, I−, F−, NO3−, CN−, OH−, H2O, HCO3− or HSO4−.

Some embodiments include A peptide platinum complex selected from the group consisting of:
a) cis-bis[AcGPSRVGGCNH₂][trans-(1R,2R)-1,2-diaminocyclohexane]platinum(II), or a pharmaceutically acceptable salt thereof, said complex or salt being in purified form;
b) cis[AcGPSRVGGCNH₂][trans-(1R,2R)-1,2-diaminocyclohexane]platinum(II), or a pharmaceutically acceptable salt thereof, said complex or salt being in purified form;c) cis-bis[AcGPSRVGGCNH₂HCl][trans-(rac)-1,2-diaminocyclohexane]platinum(II), or a pharmaceutically acceptable salt thereof, said complex or salt being in purified form; d) cis-[AcGPSRVGGCNH₂][trans-(rac)-1,2-diaminocyclohexane]platinum(II), or a pharmaceutically acceptable salt thereof, said complex or salt being in purified form;
c) cis-bis[AcGPSRVGGCNH₂-LINKER][cis-1,2-diaminocyclohexane]platinum(II), or a pharmaceutically acceptable salt thereof, said complex or salt being in purified form;
d) cis-[AcGPSRVGGCNH₂-LINKER][(cis)-1,2-diaminocyclohexane]platinum(II), or a pharmaceutically acceptable salt thereof, said complex or salt being in purified form;
e) cis-bis[AcGPSRVGGCNH₂-LINKER][trans-(1R,2R)-1,2-diaminocyclohexane]platinum(II), or a pharmaceutically acceptable salt thereof, said complex or salt being in purified form;
f) cis-[AcGPSRVGGCNH₂-LINKER][trans-(1R,2R)-1,2-diaminocyclohexane]platinum(II), or a pharmaceutically acceptable salt thereof, said complex or salt being in purified form;
g) cis-bis[AcGPSRVGGCNH₂-LINKER][trans-(rac)-1,2-diaminocyclohexane]platinum(II), or a pharmaceutically acceptable salt thereof, said complex or salt being in purified form;
h) cis-[AcGPSRVGGCNH₂-LINKER][trans-(rac)-1,2-diaminocyclohexane]platinum(II), or a pharmaceutically acceptable salt thereof, said complex or salt being in purified form;
i) cis-bis[AcGPSRVGGCN₂-LINKER][cis-1,2-diaminocyclohexane]platinum(II), or a pharmaceutically acceptable salt thereof, said complex or salt being in purified form; and
j) cis-[AcGPSRVGGCNH₂-LINKER][(cis)-1,2-diaminocyclohexane]platinum(II), or a pharmaceutically acceptable salt thereof, said complex or salt being in purified form.

Some embodiments include peptide platinum complex as above which further comprises an additional anticancer agent other than the peptide platinum complex as above or a pharmaceutically acceptable salt of the peptide platinum complex as above. In some embodiments, the additional anticancer agent is gemcitabine, capecitabine or 5-fluorouracil.

Some embodiments include a pharmaceutical composition comprising an amount of the peptide platinum complex as above or a pharmaceutically acceptable salt of the peptide platinum as above effective to treat cancer, and a pharmaceutically acceptable carrier or vehicle. Anyone of the peptide platinum complex as above further comprising an amount of an additional anticancer agent other than the aforesaid peptide platinum complex or a pharmaceutically acceptable salt of the aforesaid peptide platinum complex effective to treat cancer. Any one of the pharmaceutical composition as above wherein the additional anticancer agent is gemcitabine, capecitabine or 5-fluorouracil. Any one of the pharmaceutical composition as above comprising an amount of any one of the peptide platinum complex as above or a pharmaceutically acceptable salt of the peptide platinum complex as above effective to treat cancer, and a pharmaceutically acceptable carrier or vehicle. Any one of the pharmaceutical composition as above further comprising an amount of an additional anticancer agent other than the peptide platinum complex as above or a pharmaceutically acceptable salt of the peptide platinum complex as above effective to treat cancer.

Some embodiments include a method for treating cancer, the method comprising administering to a subject in need thereof an amount of any one of the peptide platinum complex as above or a pharmaceutically acceptable salt of the peptide platinum complex as above, effective to treat cancer. Any one of the method as above further comprising administering to said subject an additional anticancer agent which is not the peptide platinum complex as above or the pharmaceutically acceptable salt of the peptide platinum complex as above. Any one of the method as above further comprising administering to said subject an additional anticancer agent which is not the peptide platinum complex as above. Any one of the method as above wherein the additional anticancer agent is gemcitabine, capecitabine or 5-fluorouracil. Any one of the method as above wherein the cancer is pancreatic cancer, colorectal cancer or mesothelioma. Any one of the method as above, wherein the subject is a human.

Some embodiments include a kit comprising a container which contains a unit dosage form of any one of the peptide platinum complex as above or a pharmaceutically acceptable salt thereof. Any one the kit as above, further comprising a second container, the second container containing a solution for reconstitution of the peptide platinum complex. Any one the kit as above, wherein the solution is an aqueous solution. Any one the kit as above, wherein the aqueous solution comprises sodium chloride. Any one the kit as above, wherein the aqueous solution is a saline solution. Any one the kit as above, wherein the saline solution is phosphate buffered saline. Any one the kit as above, wherein the aqueous solution comprises dextrose. Any one the kit as above, wherein the dextrose solution is isotonic. Any one the kit as above, further comprising a second container, the second container containing an additional anticancer agent other than the peptide platinum complex as above or a pharmaceutically acceptable salt of the peptide platinum complex as above. Any one the kit as above, wherein the additional anticancer agent is gemcitabine, capecitabine or 5-fluorouracil. Any one the kit as above, further comprising a second container, the second container containing an antiemetic agent or a hematopoietic colony stimulating factor. Any one the kit as above, further comprising means for administering the peptide platinum complex or a pharmaceutically acceptable salt thereof to a subject.

Some embodiments include a method for making a platinum complex of formula (I),

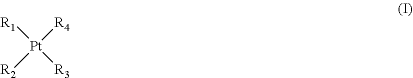

comprising allowing a complex of formula(II),

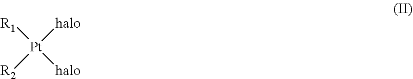

to react with at least about 2 molar equivalents of a compound of formula (II),

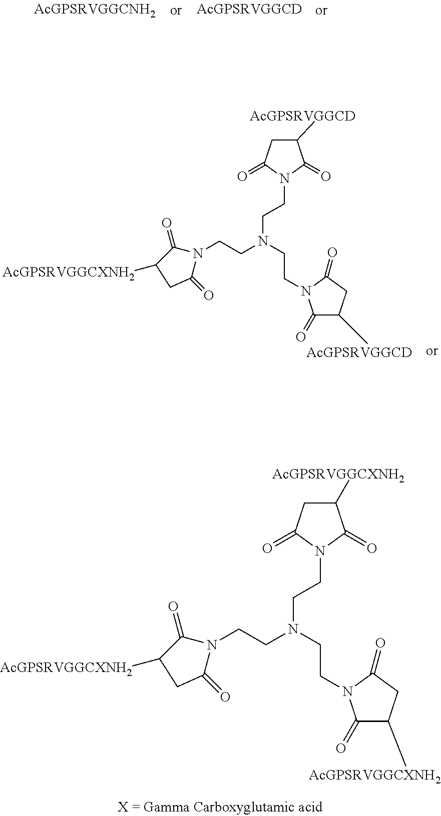

wherein
R₁ and R₂ are independently —N(R₆)₂, —NH₃⁺, or R₁ and R₂ are each —NH₂ and join through an C₂-C₆ alkylene or C₃-C₇ cycloalkylene group to form a bidentate diamine ligand;
R₃ is a peptide ligand, with the proviso that R₃ cannot be an amino acid;
R₄ is a peptide ligand, an inorganic ligand, —CN or —OC(O)R₅, with the proviso that R₄ cannot be an amino acid;
R₅ is C₁-C₂₄ alkyl;
each R₆ is independently —H, —C₁-C₆ alkyl, —C₃-C₇ cycloalkyl or -aryl; and halo is —F, —Cl, —Br, —I or —At.

Any one of the method as above, wherein R₃ is a peptide. Any one of the method as above wherein R₄ is a peptide. Any one of the method as above, wherein R₃ and R₄ are each independently a peptide. Any one of the method as above, wherein R₁ and R₂ join to form a bidentate diamine ligand. Any one of the method as above, wherein the bidentate diamine ligand is trans-R,R-1,2-diaminocyclohexane, trans-S,S-1,2-diaminocyclohexane, cis-1,2-diaminocyclohexane or 1,2-ethylenediamine. Any one of the method as above, wherein the bidendate diamine ligand is trans-R,R-1,2-diaminocyclohexane. Any one of the method as above wherein each of R₃ and R₄ comprises the peptide AcGPSRVGGCNH₂. Any one of the method as above wherein R₃ and R₄ together comprises the peptide AcGPSRVGGCNH₂ and R₃ and R₄ are attached to the same platinum moiety. Any one of the method as above wherein R₃ is AcGPSRVGGCNH₂ and R₄ is an inorganic or organic group.

Also provided by the invention is a method for treating cancer comprising administering to a subject in need of such treatment an amount of a peptide platinum complex effective to treat cancer.

The invention also includes pharmaceutical compositions that comprise an amount of a peptide platinum complex effective to treat cancer, and a pharmaceutically acceptable carrier, vehicle or excipient(s). The compositions are useful for treating cancer. The invention includes a peptide platinum complex when provided as a pharmaceutically acceptable salt.

The details of the invention are set forth in the accompanying description and examples below.

### BRIEF DESCRIPTION OF FIGURES

FIG. 1 shows a dose response curve for cell killing of the human skin malignant melanoma cell line, A375 using Onco-001

FIG. 2 shows a dose response curve for cell killing of the human skin malignant melanoma cell line, A375 using Onco-003

FIG. 3 shows a dose response curve for cell killing of the human skin malignant melanoma cell line, A375 using Onco-005

FIG. 4 shows a dose response curve for cell killing of the BEL-7402 cells using Doxorubicin.

### DETAILED DESCRIPTION OF THE INVENTION

This invention provides a novel class of coordination compounds, or more specifically peptide platinum complexes, according to Formula I, as set forth below:

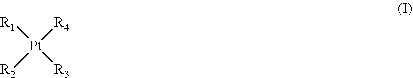

and pharmaceutically acceptable salts, solvates and hydrates thereof,
wherein
R₁ and R₂ are independently —N(R₆)₂, —NH₃⁺, or R₁ and R₂ are each —NH₂ and join through an C₂-C₆ alkylene or C₃-C₇ cycloalkylene group to form a bidentate diamine ligand;
R₃ is a peptide ligand
R₄ is a peptide ligand, an inorganic ligand, —CN or —OC(O)R₅, —Cl, —CN, —NO₃, —OH, H₂O;
R₃ and R₄ can also be the same peptide but containing two functional groups, including an amine, a carboxy or sulfhydryl, that can coordinate with Pt
   R₅ is C₁-C₂₄ alkyl; and
   each R₆ is independently —H, —C₁-C₆ alkyl, —C₃-C₇ cycloalkyl or -aryl.

In one embodiment the peptide platinum complexes are in purified form.

### Definitions

The term “C₁-C₆ alkyl” as used herein refers to a straight or branched chain, saturated or unsaturated hydrocarbon having from 1 to 6 carbon atoms. Representative C₁-C₆ alkyl groups include, but are not limited to methyl, ethyl, propyl, isopropyl, butyl, sec-butyl, tert-buty, pentyl, isopentyl, neopentyl, hexyl, isohexyl, neohexyl, ethylenyl, propylenyl, 1-butenyl, 2-butenyl, 1-pentenyl, 2-pentenyl, 1-hexenyl, 2-hexenyl, 3-hexenyl, acetylenyl, pentynyl, 1-butynyl, 2-butynyl, 1-pentynyl, 2-pentynyl, 1-hexynyl, 2-hexynyl and 3-hexynyl.

The term “alkoxy” as used herein refers to a —O—(C₁-C₆ alkyl) group. Representative alkoxy groups include, but are not limited to methoxy, ethyl, propoxy, isopropoxy, butoxy, sec-butoxy, tent-butoxy, pentyloxy, isopentyloxy, neopentyloxy, hexyloxy, isohexyloxy and neohexyloxy.

The term “C₁-C₂₄ alkyl” as used herein refers to a straight chain or branched, saturated or unsaturated hydrocarbon having from 1 to 24 carbon atoms. Representative C₁-C₂₄ alkyl groups include, but are not limited to, methyl, ethyl, propyl, butyl, pentyl, hexyl, heptyl, octyl, nonyl, decyl, undecyl, dodecyl, isopropyl, isobutyl, sec-butyl and tent-butyl, isopentyl, neopentyl, isohexyl, neohexyl, isoheptyl, neoheptyl, isooctyl, neooctyl, isononyl, neononyl, isodecyl, neodecyl, myristoyl, oleoyl, palmitoyl, stearoyl, lauroyl and caproyl. A C₁-C₂₄ alkyl group can be unsubstituted or optionally substituted with one or more —C₁-C₆ alkyl, —C₃-C₇ cycloalkyl, -alkoxy, -aryl, -heterocyclic, -halo, —CN, —COOH, —COOR₆, —OC(O)R₆, —NH₂, —C(O)R₆, —CHO, —NHR₆, N(R₆)₂, —NHC(O)R₆ or —C(O)NHR₆ groups wherein R₆ is —H, —C₁-C₆ alkyl, —C₃-C₇ cycloalkyl or -aryl.

As used herein, the term “alkylcarboxylate or alkylcarboxylato” refers to a group having the structure:

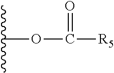

wherein R₅ is a C₁-C₂₄ alkyl group.

“C₂-C₆ alkylene” refers to a divalent, straight or branched chain, saturated hydrocarbon having from 2 to 6 carbon atoms.

The term “aryl” as used herein refers to a phenyl group or a naphthyl group.

The term “bidentate diamine ligand” as used herein refers to ligands of the general formula:

NH₂—X—NH₂

wherein X is a C₂-C₆ alkylene or C₃-C₇ cycloalkylene group which links the two NH₂ groups. Such a bidentate ligand coordinates to the platinum via the two NH₂ groups, each of which occupies a separate coordination site on the metal. A bidentate diamine ligand can be chiral or achiral. Representative bidentate diamine ligands include, but are not limited to, trans-R,R-1,2-diaminocyclohexane, trans-S,S-1,2-diaminocy clohexane, cis-1,2-diaminocyclohexane and 1,2-ethylenediamine.

As used herein, the term “linker” is used to refer to a covalent means of connecting a peptide and a platinum moiety. A linker serves not only a connecting function but also minimizes steric hindrance among the platinum peptide complex and the target as the peptide binds its target. In some instances, the linker can be designed in a such a manner to facilitate dissociation of the peptide from the platinum moiety, once the construct has been internalized into the cell. For example, use of a succinyl or a glutaryl linker will result in release through esterases/amidases action (Karampelas et al., Bioconjugate Chem., 2014, 25, 813-823; Sun et al., Curr. Drug Delivery 2011, 8, 2-10), while using para-amino benzyl alcohol or a linker with an oxime bond will yield release upon 1,6 elimination or hydrolysis in acidic pH, respectively (Zhang et al. , Eur. J. Med. Chem. 2017,139, 542-563; Szabo, I., et al., Bioconjuigate Chem 2009, 20, 20656-20665).

The term “C₃-C₇ cycloalkyl” as used herein is a 3-, 4- 5-, 6- or 7-membered saturated or unsaturated non-aromatic carbocyclic ring. Representative cycloalkyls include, but are not limited to, cyclopropyl, cyclobutyl, cyclopentyl, cyclopentadienyl, cyclohexyl, cyclohexenyl, cycloheptyl, cycloheptanyl, 1,3-cyclohexadienyl, -1,4-cyclohexadienyl, -1,3-cycloheptadienyl, and -1,3,5-cycloheptatrienyl.

“C₃-C₇ cycloalkylene” refers to a 3-, 4- 5-, 6- or 7-membered divalent, saturated or unsaturated non-aromatic carbocyclic ring. Cisplatin, PtCl2(NH3)2, is a coordination complex of platinum(II) with two chloride and two ammonia ligands. It is a commonly-used anticancer drug and has the following molecular structure.

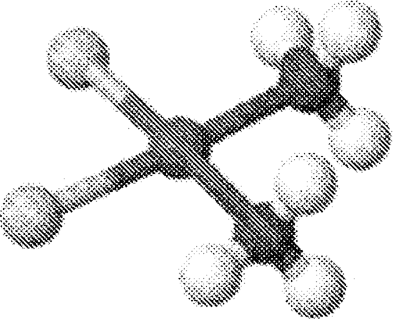

The term “coordinate complex” refers to a molecule which contains a central atom or ion, which is usually metallic and is called the coordination center, and a surrounding array of bound molecules or ions, that are in turn known as ligands or complexing agents. Lawrance, Geoffrey A. (2010). Introduction to Coordination Chemistry. Wiley. doi:10.1002/9780470687123. ISBN 9780470687123; IUPAC, Compendium of Chemical Terminology, 2nd ed. (the “Gold Book”) (1997). Online corrected version: (2006—) “complex”. doi:10.1351/goldbook.C01203; IUPAC, Compendium of Chemical Terminology, 2nd ed. (the “Gold Book”) (1997). Online corrected version: (2006—) “coordination entity”. doi:10.1351/goldbook.C01330.

A coordination complex whose center is a metal atom is called a metal complex. The central atom or ion, together with all ligands, comprise the coordination sphere. The central atoms or ion and the donor atoms comprise the first coordination sphere. A coordination complex is the product of a Lewis acid-base reaction in which neutral molecules or anions (called ligands) bond to a central metal atom (or ion) by coordinate covalent bonds. Ligands are Lewis bases - they contain at least one pair of electrons to donate to a metal atom/ion. Ligands are also called complexing agents. Metal atoms/ions are Lewis acids - they can accept pairs of electrons from Lewis bases. Within a ligand, the atom that is directly bonded to the metal atom/ion is called the donor atom. A coordinate covalent bond is a covalent bond in which one atom (i.e., the donor atom) supplies both electrons. This type of bonding is different from a normal covalent bond in which each atom supplies one electron. If the coordination complex carries a net charge, the complex is called a complex ion. Compounds that contain a coordination complex are called coordination compounds. Coordination compounds and complexes are distinct chemical species - their properties and behavior are different from the metal atom/ion and ligands from which they are composed. Coordination refers to the “coordinate covalent bonds” (dipolar bonds) between the ligands and the central atom.

The term “functional group” refers to ion or molecule that binds to the central metal atom

The term “halo” as used herein refers to —F, —Cl, —Br or —I.

The term “inorganic ligand” as used herein refers to a ligand that does not comprise a carbon-containing organic functional group. Representative examples of inorganic ligands include, but are not limited to, Cl⁻, Br⁻, I⁻, F⁻, NO₃⁻, OH⁻, H₂O, HCO₃⁻ and HSO₄⁻.

The term “peptide” refers to an amino acid oligomer which is composed of from 5 up to and including 30 amino acid residues, including all lengths between 5 and 30, for example 7 to 25 residues, 8 to 20 residues, 9 to 18 residues, and 10 to 15 residues, and all peptide lengths between these ranges. “Peptide moiety” refers to a single peptide, a multimer of the same peptide or multiple and different peptides. “Peptide ligand” refers to a peptide molecule coordinating with platinum.

The term “peptide platinum complex” as used herein, refers to a tetracoordinate platinum complex of formula (I) as described herein. In a preferred embodiment, a peptide platinum complex is in purified form.

As used herein, the term “purified” means that when isolated (e.g., from other components of a synthetic organic chemical reaction mixture), the isolate contains at least 50%, at least 55%, at least 60%, at least 65%, at least 70%, at least 75%, at least 80%, at least 85%, at least 90%, at least 95% or at least 98% of a peptide platinum complex of the invention by weight of the isolate. In a preferred embodiment, the isolate contains at least 95% of a peptide platinum complex of the invention by weight of the isolate.

The following abbreviations are used herein and have the indicated definitions: DACH is 1,2-diaminocyclohexane; ACN is acetonitrile; EtOH is ethyl alcohol; HPLC is high pressure liquid chromatography; peptide is a short chain of single amino acids linked by amide (peptide) bonds.

It should be noted that any references herein to a product by trade name is intended to include at least the corresponding generic product/name currently associated with the trade name and equivalent and similar products

### Peptide Platinum Complexes

The peptide platinum complexes of the invention are tetracoordinate platinum (II) complexes in which two adjacent coordination sites (represented by R₁ and R₂ in Formula I) are independently occupied by an amine or an ammine (NH₃⁺) ligand, or alternately, R₁ and R₂ together represent a single bidentate diamine ligand. The third coordination site (R₃ of Formula I) is occupied by a peptide ligand, while the final coordination site (R₄ of Formula I) can be occupied by a peptide ligand, an inorganic ligand or an organic ligand.

The present invention further encompasses pharmaceutically acceptable salts of the peptide platinum complexes of the invention, including both organic and inorganic salts of the peptide platinum complexes of the invention. The peptide platinum complexes of the invention contain at least one amino group, and accordingly, it is possible to form acid addition salts with an amino group of a peptide platinum complex of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion can be any organic or inorganic moiety that stabilizes a charge, which may be present on a peptide platinum complex of the invention. Furthermore, a pharmaceutically acceptable salt may have more than one charged atom in its structure. In instances where multiple charged atoms are part of the pharmaceutically acceptable salt, said salt can have multiple counterions. Hence, a pharmaceutically acceptable salt of a peptide platinum complex can have one or more charged atoms and/or one or more counterions.

### Amine/Ammine Ligands

R₁ and R₂ are independently an amine or ammine (NH₃⁺) ligand. The amine ligands of the invention are represented by the formula —N(R₆)₂ where each R₆ is independently —H, —C₁-C₆ alkyl, —C₃-C₇ cycloalkyl or -aryl. In an alternate embodiment, R₁ and R₂ are each —NH₂ and join through a C₂-C₆ alkylene or C₃-C₇ cycloalkylene group to form a bidentate diamine ligand. Bidentate diamine ligands useful in the invention include, but are not limited to, trans-R,R-1,2-diaminocyclohexane, trans-S,S-1,2-diaminocyclohexane, cis-1,2-diaminocyclohexane and 1,2-ethylenediamine.

In one embodiment, a peptide platinum complex comprises a bidentate diamine ligand.

In a specific embodiment, a peptide platinum complex comprises a bidentate 1,2-ethylenediamine ligand.

In a particular embodiment, a peptide platinum complex comprises a bidentate 1,2-diaminocyclohexane ligand.

In a preferred embodiment, a peptide platinum complex comprises a bidentate trans-R,R-1,2-diaminocyclohexane ligand.

### Peptide Ligands

Peptides are useful herein as addition moieties to platinum to form the peptide platinum complexes of the invention, which complexes comprise a tetracoordinate platinum complex having at least one peptide ligand.

A peptide useful according to the invention will be capable of binding to an antigen present on the surface or within the cancer cell. Such a peptide will possess a high degree of specificity for its target. The affinity of a given peptide for its target can be assessed using a binding assay by means of a radioassay, ELISA, or surface plasmon resonance (SPR) and will have a binding affinity in the range of high micromolar to sub-nanomolar. Specificity of a given peptide can be assessed using a cell-based assay, ELISA or SPR. A peptide useful according to the invention is composed of amino acids and therefore each peptide has an amino group and a carboxy group, or possibly a sulfhydryl, from which to connect the peptide to the platinum compound.

A peptide useful according to the invention for targeting breast cancer may contain or consist of the sequence CRXXRXXXC (where X can be any amino acid other than C, R, W or Y; SEQ ID NO: 1), RGX1PAYX2GRFL (where X1 can be D or E and X2 can be Q or N; SEQ ID NO: 2) or PXLNVSP (where X is any amino acid, except for C, W and Y; SEQ ID NO: 3). A peptide for targeting melanoma may contain or consist of the sequence motif PRP, WRP and/or S/ThXh(S/T)WXPP (where S/T implies that either will work, h represents a hydrophobic amino acid, such as I, L, A or V and X can be any except C and W; SEQ ID NOs: 151, 152, 153 and 154). For pancreatic cancer, a tumor targeting peptide may contain or consist of the following consensus sequence: PXIXIT (where X can be any amino acid, except C, Y and W; SEQ ID NO: 4). For glioblastoma, a cancer targeting peptide will contain or consist of the consensus sequence VD/GLPE/THXX (where D/G implies either D or G and similarly for E/T; X can be any amino acid except for C, W and Y; SEQ ID NOs: 155, 156, 157 and 158). A targeting peptide for lung cancer may contain or consist of the sequence XPWXEXXYX (where X is any amino acid except for W, Y and C; SEQ ID NO: 5).

See, for example, targeting peptides set forth in the following citations and Table 1.

TABLE 1
List of Targeting (homing) peptides to various
cancer targets

| Cancer Target | Peptide sequence |
|---|---|
| Breast | CSNIDARAC (SEQ ID NO: 6) |
| | CDPSRGKNC (SEQ ID NO: 7) |
| | CPSDLKDAC (SEQ ID NO: 8) |
| | CRTTRGTKC (SEQ ID NO: 9) |
| | CRMTRNKPC (SEQ ID NO: 10) |
| | CRVSRQNKC (SEQ ID NO: 11) |
| | CAKIDPELC (SEQ ID NO: 12) |
| | CGGERGKSC (SEQ ID NO: 13) |
| | KGVSLSYR (SEQ ID NO: 14) |
| | RTRYED (SEQ ID NO: 15) |
| | GMMYRS (SEQ ID NO: 16) |
| | RWRTNF (SEQ ID NO: 17) |
| | RIPLEM (SEQ ID NO: 18) |
| | QFDEPR (SEQ ID NO: 19) |
| | TSAVRT (SEQ ID NO: 20) |
| | GLWQGP (SEQ ID NO: 21) |
| | QCTGRF (SEQ ID NO: 22) |
| | LPGMMG (SEQ ID NO: 23) |
| | DVGTTE (SEQ ID NO: 24) |
| | TDLGAM (SEQ ID NO: 25) |
| | DSNAES (SEQ ID NO: 26) |
| | ITDMAA (SEQ ID NO: 27) |
| | WRPCES (SEQ ID NO: 28) |
| | WRNTIA (SEQ ID NO: 29) |
| | IDKQLE (SEQ ID NO: 30) |
| | FMEIET (SEQ ID NO: 31) |
| | HEVVAG (SEQ ID NO: 32) |
| | GGHTRQ (SEQ ID NO: 33) |
| | INGKVT (SEQ ID NO: 34) |
| | VPWXEPAYQRFL (SEQ ID NO: 35) |
| | GRDS (SEQ ID NO: 36) |
| | RGEPAYQRFL (SEQ ID NO: 37) |
| | RGDPAYQRFL (SEQ ID NO: 38) |
| | WXEPAYQGRFL (SEQ ID NO: 39) |
| | WXEPAYNGRFL (SEQ ID NO: 40) |
| | RGEPAYQGRFL (SEQ ID NO: 41) |
| | RGDPAYQGRFL (SEQ ID NO: 42) |
| | RGEPAYNGRFL (SEQ ID NO: 43) |
| | RGDPAYNGRFL (SEQ ID NO: 44) |
| | WXEPAYQRFL (SEQ ID NO: 45) |
| | AXEPAYQRFL (SEQ ID NO: 46) |
| | WAEPAYQRFL (SEQ ID NO: 47) |
| | WXAPAYQRFL (SEQ ID NO: 48) |
| | WXEAAYQRFL (SEQ ID NO: 49) |
| | WXEPAAQRFL (SEQ ID NO: 50) |
| | WXEPAYARFL (SEQ ID NO: 51) |
| | WXEPAYQAFL (SEQ ID NO: 52) |
| | WXEPAYQAAL (SEQ ID NO: 53) |
| | WXEPAYQAFA (SEQ ID NO: 54) |
| | EXEPAYQRFL (SEQ ID NO: 56) |
| | LXEPAYQRFL (SEQ ID NO: 56) |
| | KXEPAYQRFL (SEQ ID NO: 57) |
| Melanoma | PRPGAPLAGSWPGTS (SEQ ID NO: 58) |
| | ADGAPRPGAPLA (SEQ ID NO: 59) |
| | DRWRPALPVVLFPLH (SEQ ID NO: 60) |
| | ASSSYPLIHWRPWAR (SEQ ID NO: 61) |
| | DRWRPALP (SEQ ID NO: 62) |
| | IHWRPWAR (SEQ ID NO: 63) |
| | AAEWLDAFFVRHVDR (SEQ ID NO: 64) |
| | GDVWLFLTSTSHFAR (SEQ ID NO: 65) |
| | GCSVSSVGALCTHV (SEQ ID NO: 66) |
| | APCCSHLDASPFQRP (SEQ ID NO: 67) |
| | AQSNFVTWGYNVAV (SEQ ID NO: 68) |
| | RASDVGSDVVPRYPF (SEQ ID NO: 69) |
| | CRGDCGGKWCFRVCYRGICYRRCR |
| | (SEQ ID NO: 70) |
| | TAASGVRSMH (SEQ ID NO: 71) |
| | LTLRWVGLMS (SEQ ID NO: 72) |
| | CVLNGRMEC (SEQ ID NO: 73) |
| | KKEKDIMKKTI (SEQ ID NO: 74) |
| | GRGDSPK (SEQ ID NO: 75) |
| | CRGDGWC (SEQ ID NO: 76) |
| Prostate | CGRRAGGSC (SEQ ID NO: 77) |
| | CRGDCGGKWCFRVCYRGICYRRCR |
| | (SEQ ID NO: 78) |
| | RPARPAR (SEQ ID NO: 79) |
| | CRGDKGPDC (SEQ ID NO: 80) |
| | CRGDRGPDC (SEQ ID NO: 81) |
| | CRGDKTTNC (SEQ ID NO: 82) |
| | CRGDHAGDC (SEQ ID NO: 83) |
| | CRGDHGVEC (SEQ ID NO: 84) |
| | CGRGDNLPC (SEQ ID NO: 85) |
| | CGRGDNLAC (SEQ ID NO: 86) |
| | CEKRGDNLC (SEQ ID NO: 87) |
| Sarcoma | NGRAHA (SEQ ID NO: 88) |
| | KDEPQRRSARLSAKPAPPKPEPKPKKAPAKK |
| | (SEQ ID NO: 89) |
| | CREKA (SEQ ID NO: 90) |
| | TDCTPSRCT (SEQ ID NO: 91) |
| | SWCQFEKCL (SEQ ID NO: 92) |
| | VPCRFKQCW (SEQ ID NO: 93) |
| | CTAMRNTDC (SEQ ID NO: 94) |
| | CRESLKNC (SEQ ID NO: 95) |
| | CMEMGVKC (SEQ ID NO: 96) |
| Lung | SYDILKPNPQRL (SEQ ID NO: 97) |
| | SHGKPPSFSPYT (SEQ ID NO: 98) |
| | CLSYYPSYC (SEQ ID NO: 99) |
| | RLQLKL (SEQ ID NO: 100) |
| | GHGKHKNK (SEQ ID NO: 101) |
| | TDSILRSYDWTY (SEQ ID NO: 102) |
| | DMPKOLLAPWYY (SEQ ID NO: 103) |
| | SYPLSFLGPLIS (SEQ ID NO: 104) |
| Colorectal | CPKSNNGVC (SEQ ID NO: 105) |
| | CKTPNGHLC (SEQ ID NO: 106) |
| | CQSISTAHC (SEQ ID NO: 107) |
| | CNDDVPNKC (SEQ ID NO: 108) |
| | CEIPGKVVC (SEQ ID NO: 109) |
| | CLRTPANHC (SEQ ID NO: 110) |

Additional peptides, and their properties are described in Le Joncour V1, Laakkonen, Bioorg Med Chem. 2018 Jun 1;26(10):2797-2806. doi: 10.1016/j.bmc.2017.08.052. Epub 2017 Sep 1, and are set forth in Table 2:

TABLE 2
Previously Characterized Targeting peptides

| Peptide | | | |
|---|---|---|---|
| Name | Sequence | Origin | Target |
| Octreotide | DFCFDWKTCT-ol (SEQ ID NO: 111) | n | SSTR |
| RC160 | DFCYDWKVCW (SEQ ID NO: 112) | n | SSTR |
| Bombesin | YQRLGNQWAVGHLM (SEQ ID NO: | n | GRPR |
| | 113) | | |
| PSAP-peptide | DWLPK (SEQ ID NO: 114) | n | apoptotic cascade, ? |
| NT21MP | LGASWHRPDKCCLGYQKRPLP (SEQ | n | CXCR4 |
| | ID NO: 115) | | |
| Nef-M1 | NAACAWLEAQ (SEQ ID NO: 116) | n | CXCR4 |
| Peptide R | RACRFFC (SEQ ID NO: 117) | n | CXCR4 |
| Pentixafor | DY-[NMe]DOrn-R-2Nal-G (SEQ ID NO: | n | CXCR4 |
| | 118) | | |
| pHLIP | ACEQNPIYWARYADWLFTTPLLLLDL | n | pH, cell membrane |
| | ALLVDADET (SEQ ID NO: 119) | | |
| L-zipper | VSSLESKVSSLESKVSKLESKKSKLESK | n | temperature |
| peptide | VSKLESKVSSLESK (SEQ ID NO: 120) | | |
| ELP | VPGXG (SEQ ID NO: 121) | n | temperature |
| x-MSH | Modifications of the x-MSH | n | MC1R |
| mimics | sequence([Ac-N]LDHDFRWGL) (SEQ | | |
| | ID NO: 122) | | |
| GZP | AGGIEFAD (SEQ ID NO: 123) | n | granzyme B |
| CRGD | RGDDYK (SEQ ID NO: 124) | s | xVb3 |
| EETI 2.5 F | GCPRPRGDNPPLTCSQDSDCLAGCVC | s | integrins |
| (knottin) | GPNGFCG (SEQ ID NO: 125) | | |
| NGR | CNGRC (SEQ ID NO: 126) | s | APN (CD13) |
| SP2012 | LRRFSTMPFMF-Abu-NINNV-Abu-NF | s | b1 integrins |
| | (SEQ ID NO: 127) | | |
| AARP | CTTHWGFTLC (SEQ ID NO: 128) | s | MMP2/9 + blood |
| | | | vessels |
| CK | CVNHPAFAC-HTMYYHHYQHHL | s | Sonic hedgehog + |
| | (SEQ ID NO: 129) | | VEGFR2 |
| LyP-1 | CGNKRTRGC (SEQ ID NO: 130) | s | p32 |
| AGR | CAGRRSAYC (SEQ ID NO: 131) | s | prostate cancer |
| | | | lymphatics |
| REA | CREAGRKAC (SEQ ID NO: 132) | s | pre-malignant tumor |
| | | | lymphatics |
| LSD | CLSDGKRKC (SEQ ID NO: 133) | s | tumor lymphatics |
| IRGD | CRGDKGPDC (SEQ ID NO: 134) | s | xVb3 + NRP-1 |
| iPhage/pen | [M13]-RQIKIWFQNRRMKWKK (SEQ | n | cell cytoplasm |
| | ID NO: 135) | | |
| M2pep | YEQDPWGVKWWY (SEQ ID NO: 136) | s | M2/TAM |
| Coop | CGLSGLGVA (SEQ ID NO: 137) | s | MDGI |
| CLT-1 | CGLIIQKNEC (SEQ ID NO: 138) | s | fibrosis |
| Pep-1 L | CGEMGWVRC (SEQ ID NO: 139) | s | IL13RA2 |
| Angiopep-2 | TFFYGGSRGKRNNFKTEEY (SEQ ID | n | LRP-1 |
| | NO: 140) | | |
| Angiopep-7 | TFFYGGSRGRRNNFRTEEY (SEQ ID | n | — |
| | NO: 141) | | |
| FHK | FHKHKSPALSPV (SEQ ID NO: 142) | s | tenascin-c |
| tLyP-1 | CGNKRTR (SEQ ID NO: 143) | s | NRP-1 |
| Cilengitide | CRGDf[N-Me]V (SEQ ID NO: 144) | s | integrins |

Key: n, peptide sequence based on the natural ligand; s, synthetic sequence (isolated for instance from phage displayed peptides libraries); X in a sequence means any amino acid residue, ? means that the exact targeted protein/receptor is unknown. SSTR, somatostatin receptor; GRPR, gastrin releasing peptide receptor; PSAP, presenilin-associated protein; CXCR4, stromal-derived factor receptor; pHLIP, pH low insertion peptide; ELP, elastin-like peptide; x-MSH, x-melanocyte-stimulating hormone; MC1R, melanocortin 1 receptor; GZP, granzyme B peptide; APN (CD13), aminopeptidase N; Abu, L-x-amino-n-butyric acid; MMP, metalloprotease; VEGFR2, vascular endothelial growth factor receptor 2; p32, replication protein A; NRP-1, neuropilin receptor-1; pen, penetratin; TAM, tumor-associated macrophage; MDGI, mammary-derived growth inhibitor; LRP-1, low density lipoprotein receptor-related protein-1.

Peptide ligands useful in the present invention include, but are not limited to, natural and synthetic peptides with linear and cyclic structure. Peptides of the invention are composed of either natural or synthetic amino acids, which are described in Lehninger Principles of Biochemistry by David L. Nelson and Michael M. Cox 7th ed., VetBooks, W. H. Freeman and Company, New York.

In one embodiment, a peptide platinum complex has a single peptide ligand. This is especially the case if the peptide contains functional groups for bidentate coordination as is the case for aspartic acid, glutamic acid and gamma-carboxyglutamic acid through coordination of the dicarboxylate or else through the free N-terminus of the peptide and amine of lysine within the peptide or sulfhydryl of a cysteine within the peptide.

In another embodiment, a peptide platinum complex has first and second peptide ligands, which may be the same or different. For peptides with a single functional group that can ligate with the platinum, then two peptides, whether the same or different, are likely to coordinate, if there is no steric hindrance between the two peptides.

In another embodiment, a single peptide ligand is attached to two platinum moieties.

In another embodiment, one peptide platinum complex has one peptide ligand, which is attached to the same platinum moiety.

In one embodiment, the peptide ligand is connecting to platinum moiety through a carboxylic or an amino group.

In another embodiment, the peptide ligand has the carboxylic group blocked and connects to the platinum moiety exclusively through an amino group.

In another embodiment, the peptide ligand has the amino group blocked and connects to the platinum moiety exclusively through a carboxylic group.

In another embodiment, the peptide ligand has the amino group blocked and connects to the platinum moiety exclusively through two carboxylic groups of the same peptide.

In a specific embodiment, the peptide ligand is connected to the platinum moiety through a carboxylic, amino or a thio group.

In a preferred embodiment, the amino group is blocked, and the peptide ligand is connected to the platinum moiety exclusively through a thio group.

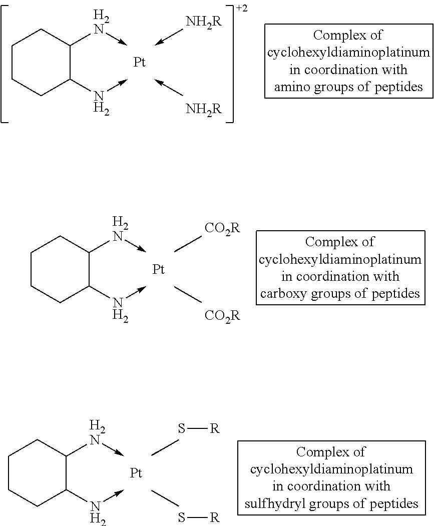

Coordination of peptides to platinum can take place through the amine, the sulfhydryl or the carboxy group. It is preferable to selectively coordinate through the carboxy group, as this is a better leaving group. Dissociation of the peptide from the platinum is necessary once the molecule is inside the cell, thus permitting the platinum moiety to then intercalate into the cellular DNA.

Some peptides useful in this invention are connected to the platinum moiety through a thio group and the peptide portion of the complex has a high binding affinity (˜50 μM to 1 nM) for a binding partner (receptor) on the cancer cell; for low binding the affinity would be >1 mM.

### Other Ligands

The peptide platinum complexes can also comprise non-amino and/or non-peptide ligands.

Other ligands useful in the invention include, but are not limited to, inorganic ligands, including, but not limited to, Cl⁻, Br⁻, I⁻, NO₃⁻, OH⁻, H₂O, HCO₃⁻, CO₃⁼ and HSO₄⁻ and organic ligands, including but not limited to, —CN and alkylcarboxylato groups of the formula —OC(O)R₅, wherein R₅ is C₁-C₂₄ alkyl.

Additional ligands useful according to the invention include, but are not limited to, antibodies, antibody fragments containing an antibody variable region composed of two VH and two VL chains, and single domain antibodies, such as a VH and/or a VL.

### Preparation of Peptide Platinum Complexes

Peptide platinum complexes of formula (I) can be prepared via the synthetic procedure outlined below in Scheme 1. It will be apparent to one skilled in the art how to prepare the scope of the peptide platinum complexes of the invention by choice of proper and relevant starting materials, synthetic intermediates and reagents.

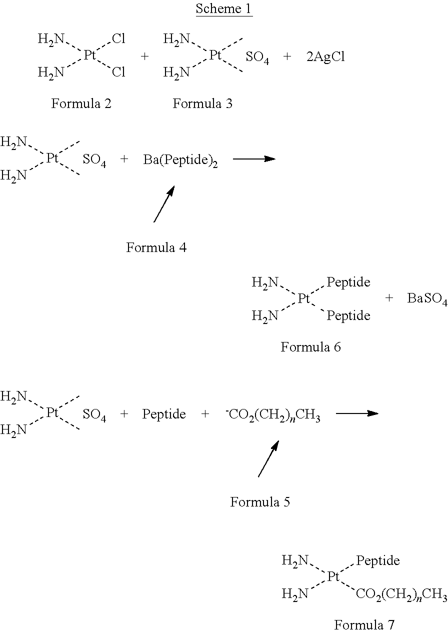

In a typical procedure, diaminodichloroplatinum complex of formula 2 is treated with silver sulfate and water to provide the intermediate diamino sulfatoplatinum (II) monohydrate of formula 3. Intermediate 3 can then be reacted with a stoichiometric excess of a reactive peptide of formula 4, to provide the diamino-bis-peptide platinum complex of diaminocyclohexane]diiodoplatinum(II) formula 6. In a specific embodiment, instead of reacting intermediate 3 with a stoichiometric excess of a single peptide, intermediate 3 may be reacted simultaneously with one equivalent each of two different peptides in order to provide a peptide platinum complex having two different peptide ligands. The complex of formula 3 can alternately be reacted with a sub-stoichiometric amount of a peptide, such as 4, in the presence of an excess of another reactive ligand, such as the alkali metal salt of an inorganic ligand or the alkali metal salt of an alkylcarboxylate 5, to yield the platinum complex of formula 7, which then contains both peptide and non-peptide ligation.

In an alternate embodiment, peptide platinum complexes of formula (I) can be prepared via the synthetic procedure outlined below in Scheme 2.

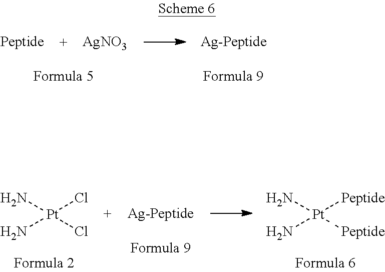

In Scheme 2, two equivalents of the peptide of formula 5 are reacted with two equivalents of AgNO₃ to provide two equivalents of the peptide of formula 9. Two equivalents of peptide of formula 9 are then reacted with one equivalent of platinum complex 2 in chloroform to yield the diamino-bis-peptide platinum complex of formula 6.

In a specific embodiment of Scheme 2, the generic bidentate platinum complex 2 is cis-[trans-(1R,2R)-1,2-diaminocyclohexane]diiodoplatinum(II) (10), the generic sodium salt of peptide 5 is the sodium salt of AcGPSRVGGCNH₂Na (SEQ ID NO: 145) (13), and the complex formed of complex 10 and silver complex 9 is cis-bis[AcGPSRVGGCNH₂][trans-(1R,2R)-1,2-diaminocyclohexane]platinum(II), (14).

### General Procedure for the Preparation of a Diaminodichloro Platinum Complex 2 of Formula 2

An approximately 0.5M solution of a diamine 1 in water is added slowly to a filtered solution of K₂PtCl₄ (1.05 eq.) in water at room temperature. The resulting mixture is allowed to stand at room temperature for about 16 hours, after which time a colored precipitate may appear. The precipitate is filtered and washed with water until the filtrate is no longer reactive toward AgNO₃ and the filtrate is then washed sequentially with ethanol and acetone. The precipitate is allowed to dry in the filtration funnel for about 1 hour and is then dried in vacuo to provide a complex of formula 2.

### General Procedure for the Preparation of a Diamino Sulfatoplatinum Complex of Formula 3

An approximately 0.03M solution of Ag₂SO₄ (about 0.97 eq.) in water is taken up in a reaction vessel that is protected from light and the resulting aqueous solution is warmed to about 40° C. The complex of formula 2 is added, and the reaction is allowed to stir for about 48 hours at room temperature. The reaction mixture is then filtered through a ˜1 cm pad of Celite and the Celite is washed with water. The filtrate is then concentrated in vacuo and the resulting solid residue is dried in vacuo to provide a complex of formula 3.

### General Procedure for the Preparation of a Diamino-Bis-Peptide Platinum Complex 6: of Formula 6

To an approximately 0.1M stirred solution of the complex of formula 3 in water is added an approximately 0.5 M solution of a peptide sodium salt 4 (about 2.0 eq.) in a mixture of H₂O:EtOH (about 8:1 by volume). The reaction mixture is stirred for about 24 hours and the resulting precipitate is filtered, washed with water and dried in vacuo to provide the complex of formula 6, which can be further purified using column chromatography or HPLC.

### General Procedure for the Preparation of a Diamino-Peptide Platinum Complex 7: of Formula 7

To an approximately 0.5M stirred solution of complex of formula 3 in water is added a solution of a peptide sodium salt 4 (about 0.5 eq.) and an alkoxycarbonyl sodium salt 5 (about 1.5 eq.) in a mixture of H₂O:EtOH (about 8:1 by volume). The reaction mixture is stirred for about 24 hours and the resulting precipitate is filtered, washed with water and dried in vacuo to provide the complex of formula 7, which can be further purified using column chromatography or HPLC.

Complexes of the invention that can be prepared using the method of Scheme 1 included, but are not limited to, AcGPSRVGGCNH₂ (SEQ ID NO: 145).

In one embodiment, the peptide platinum complex is cis-bis[AcGPSRVGGCNH₂]trans-(1R,2R)-1,2-diaminocyclohexane]platinum(II).

In a preferred embodiment, the peptide platinum complexes are in purified form.

The present invention also relates to methods for making a compound of formula (I). In one embodiment, the invention relates to a method making a compound of formula (I), comprising allowing a complex of formula (II),

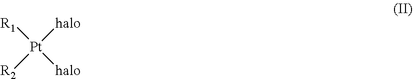

to react with at least about 2 molar equivalents of a compound of formula (III),
peptide ligands (R₃ and R₄) or —CN or —OC(O)R, —NO₃, —OH, H₂O, —CO₃ (III) where halo is —F, —Cl, —Br, —I or —At; and R₁, R₂, R₃, R₄, X¹ and X² are as defined above.

In one embodiment, the invention relates to a method for making a compound of formula (I) where R₃ is a peptide.

In one embodiment, the invention relates to a method for making a compound of formula (I) where R₄ is a peptide.

In one embodiment, the invention relates to a method for making a compound of formula (I) where R₃ and R₄ are each independently peptides

In one embodiment, the invention relates to a method for making a compound of formula (I) where R₁ and R₂ join to form a bidentate diamine ligand.

In another embodiment, the invention relates to a method for making a compound of formula (I) where R₁ and R₂ join to form a bidentate diamine ligand and R₃ and R₄ join to form a bidentate peptide ligand.

In another embodiment, the invention relates to a method for making a compound of formula (I) where R₁ and R₂ join to form a bidentate diamine ligand and the bidentate diamine ligand is trans-R,R-1,2-diaminocyclohexane, trans-S,S-1,2-diaminocyclohexane, cis-1,2-diaminocyclohexane or 1,2-ethylenediamine.

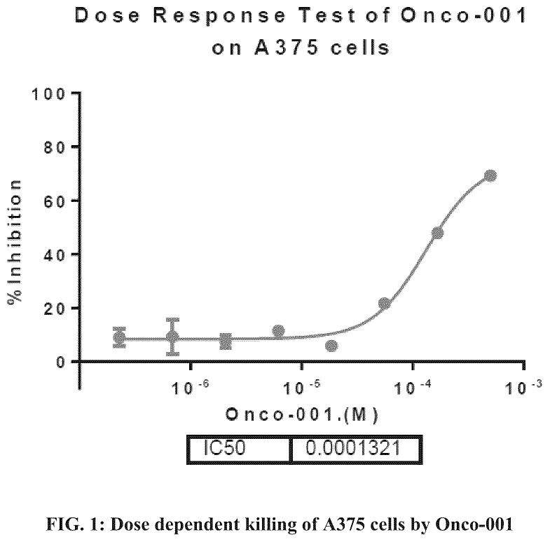

In another embodiment, the invention relates to a method for making a compound of formula (I) where R₁ and R₂ join to form trans-R,R-1,2-diaminocyclohexane, and R₃ and R₄ are each AcGPSRVGGCNH₂ (Onco-001; SEQ ID NO: 145) (14). A dose response curve for cell killing of the human skin malignant melanoma cell line, A375, is shown as FIG. 1.

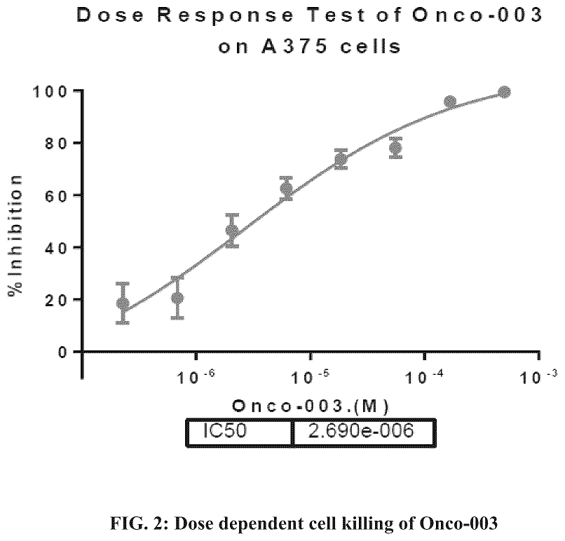

In yet another embodiment, the invention relates to a method for making a compound of formula (I) where R₁ and R₂ join to form trans-R,R-1,2-diaminocyclohexane, and R₃ and R₄ are joined by the bidentate peptide: AcGPSRVGGCD (Onco-003; SEQ ID NO: 146) (15). The dose dependent cell killing of Onco-003, using A375 cells, is displayed in FIG. 2.

In another embodiment, the invention relates to a method for making a compound of formula (I) where R₁ and R₂ join to form trans-R,R-1,2-diaminocyclohexane, and R₃ and R₄ are joined by the bidentate peptide, defined in Formula 10, below:

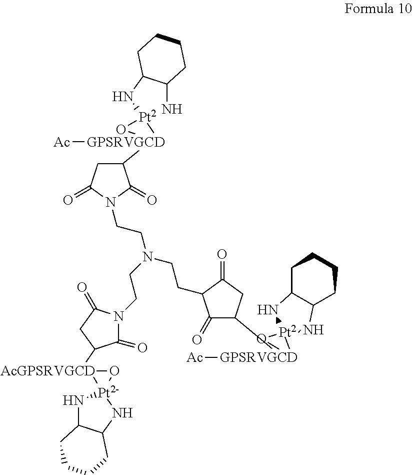

In this embodiment, one equivalent of formula 10 is linked to 3 equivalents of formula (I) where R₁ and R₂ join to form trans-R,R-1,2-diaminocyclohexane.

In a separate embodiment, the invention relates to a method for making a compound of formula (I) where R₁ and R₂ join to form trans-R,R-1,2-diaminocy clohexane, and R₃ and R₄ are joined by the peptide: Ac-GPSRVGGCD (structure provided, belowSEQ ID NO: 146).

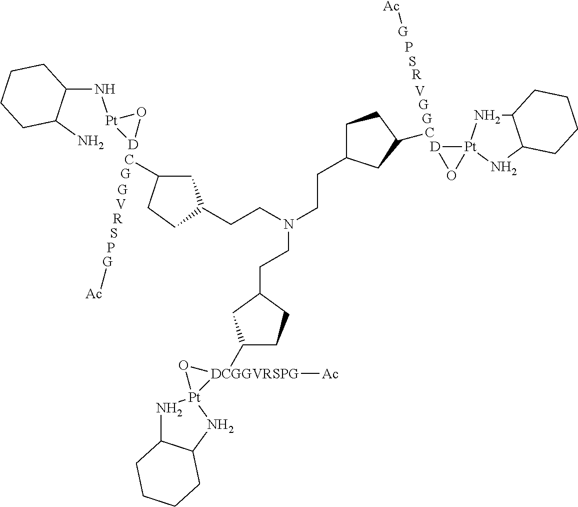

In other embodiments, the peptide complex as displayed below, as a tri-peptide species, is comprised of the peptide with sequence of AcGPSRVGGCXNH₂(SEQ ID NO: 147), where X is gamma carboxy-glutamic acid (formula 11, Onco-005). The two carboxylic moieties of the gamma carboxy-glutamic acid on each peptide coordinate with R₃ and R₄ in formula (I), resulting in three equivalent of formula (I) with one equivalent of formula 11, with structure shown below.

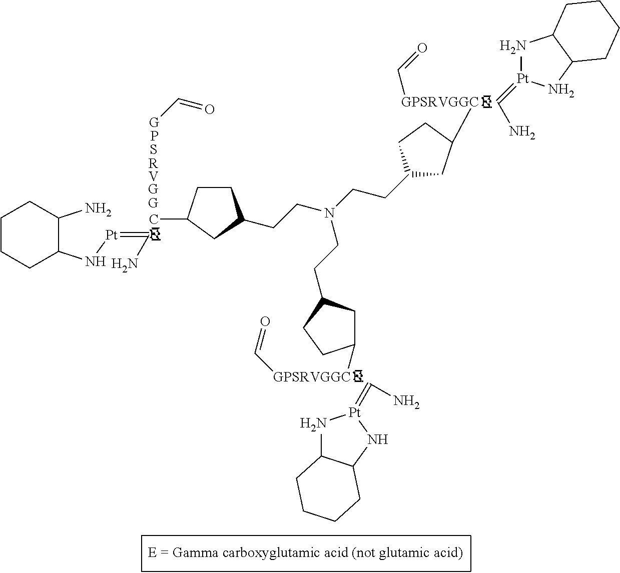

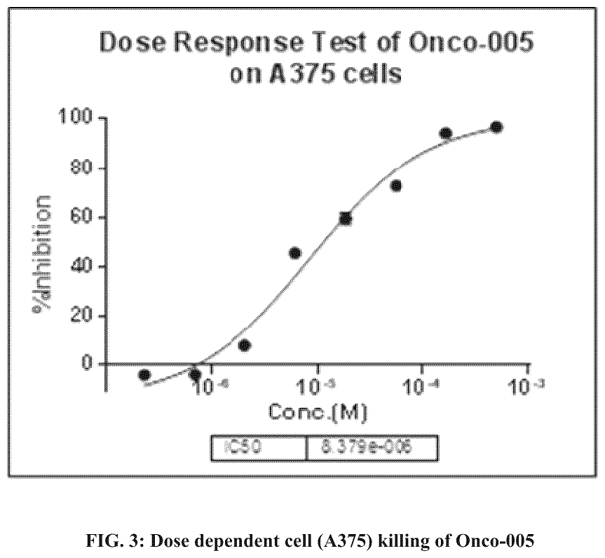

Dose dependent cell killing of Onco-005 (15), using A375 cells, is displayed in FIG. 3, below.

Other platinum (II)-peptide conjugates specific for different types of tumor are shown below.

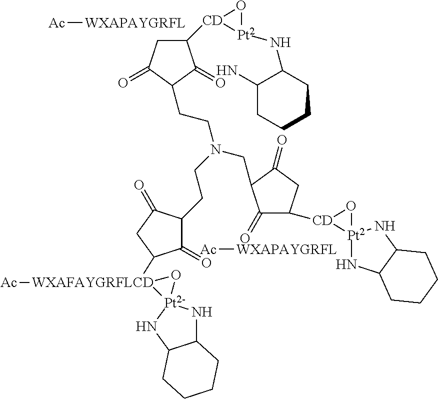

Example of a platinum (II)—peptide conjugate specific for breast cancer

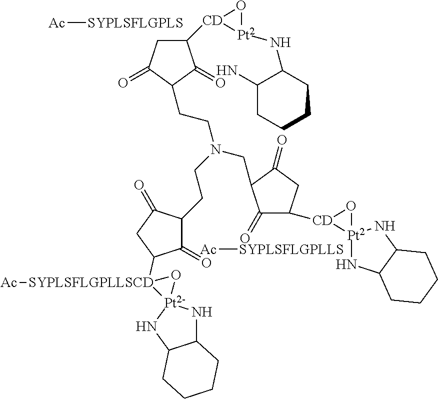

Example of a platinum (II)—peptide conjugate specific for lung cancer

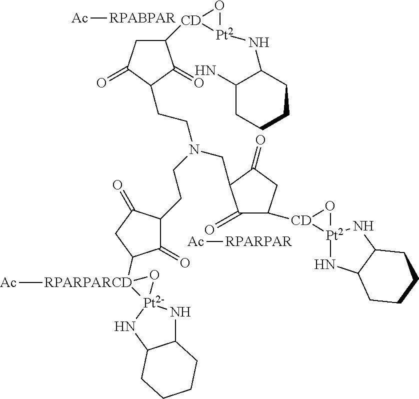

Example of a platinum (II)—peptide conjugate specific for prostate cancer

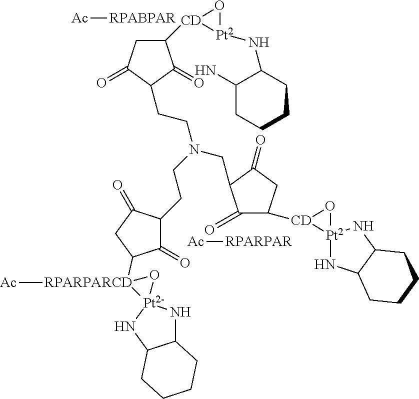

Example of a platinum (II)—peptide conjugate specific for sarcoma

While none of these platinum-peptide conjugate examples, provided above, against breast, lung, prostate and sarcoma cancers have synthesized and tested either in-vitro or in-vivo, it is, nonetheless, predicted that the anti-cancer activity of these is going to be comparable to the anti-melanoma platinum (II)-peptide conjugates (Onco-003, Onco-005), therefore displaying EC50 values ranging between 0.01 μM and 100 μM.

### Pharmaceutical Compositions and Therapeutic Administration

The present invention provides a pharmaceutical composition comprised by an effective amount of the active compound and a pharmaceutically acceptable carrier or vehicle. The pharmaceutical compositions are suitable for human or veterinary administration.

The pharmaceutical compositions of the present invention can be in any form that allows for the composition to be administered to a subject including, but not limited to a human, mammal, or non-human animal, such as a cow, horse, sheep, pig, fowl, cat, dog, mouse, rat, rabbit, guinea pig, etc., and is more preferably a mammal, and most preferably a human.

The compositions of the invention can be in the form of a solid, liquid or gas (aerosol). Typical routes of administration may include, without limitation, oral, topical, parenteral, sublingual, rectal, vaginal, ocular, and intranasal. Parenteral administration includes subcutaneous injections, intravenous, intramuscular, intraperitoneal, intrapleural, intrasternal injection or infusion techniques. Preferably, the compositions are administered parenterally, most preferably intravenously. Pharmaceutical compositions of the invention can be formulated to allow a compound of the invention to be bioavailable upon administration of the composition to a subject. Compositions can take the form of one or more dosage units, where for example, a tablet can be a single dosage unit, and a container of a compound of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment of, the use of the composition as part of a multi-drug regimen, the particular form of the compound of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is a preferred adjuvant. An injectable composition is preferably sterile.

The amount of the compound of the invention that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The pharmaceutical compositions comprise an effective amount of a compound of the invention such that a suitable dosage will be obtained. Typically, this amount is at least 0.01% of a compound of the invention by weight of the composition. When intended for oral administration, this amount can be varied to be between 0.1% and 80% by weight of the composition. Preferred oral compositions can comprise from between 4% and 50% of the compound of the invention by weight of the composition. Preferred compositions of the present invention are prepared so that a parenteral dosage unit contains from between 0.01% and 2% by weight of the compound of the invention.

The compounds of the invention can be administered in a single dose or in multiple doses.

In one embodiment, the compounds of the invention are administered in multiple doses. When administered in multiple doses, the compounds are administered with a frequency and in an amount sufficient to treat the condition. In one embodiment, the frequency of administration ranges from once a day up to about once every eight weeks. In another embodiment, the frequency of administration ranges from about once a week up to about once every six weeks. In another embodiment, the frequency of administration ranges from about once every three weeks up to about once every four weeks.

Generally, the dosage of a compound of the invention administered to a subject is in the range of 0.1 to 50 mg/kg, and more typically in the range of 0.1 mg/kg to 100 mg/kg, of the subject's body weight. In one embodiment, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the subject's body weight.

The compounds of the invention can be administered by any convenient route, for example by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering a compound of the invention. In certain embodiments, more than one compound of the invention is administered to a subject. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The preferred mode of administration is left to the discretion of the practitioner and will depend in-part upon the site of the medical condition (such as the site of cancer, a cancerous tumor or a pre-cancerous condition).

In one embodiment, the compounds of the invention are administered parenterally.

In a preferred embodiment, the compounds of the invention are administered intravenously.

In specific embodiments, it can be desirable to administer one or more compounds of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor, or precancerous tissue. In certain embodiments, it can be desirable to introduce one or more compounds of the invention into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and carriers such as triglycerides.

In yet another embodiment, the compounds of the invention can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the compounds of the invention, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) can be used.

The term “carrier” refers to a diluent, adjuvant or excipient, with which a compound of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the compounds of the invention and pharmaceutically acceptable carriers are sterile. Water is a preferred carrier when the compound of the invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E. W. Martin.

Sustained or directed release compositions that can be formulated can include, but are not limited to, the liposomal (lipid nanoparticles), polymeric nanoparticles, prepared with PLGA and other biocompatible polymers, and micellar platinum complexes of the invention, virus-like particles and other formulations where a peptide platinum complex of the invention is protected with differentially degradable coatings, e.g., by microencapsulation, multiple coatings, etc. It is also possible to freeze-dry the compositions and use the lyophilizates obtained, for example, for the preparation of products for injection.

In a preferred embodiment, the peptide platinum complexes of the invention are delivered in solution. In a particularly preferred embodiment, the solution is containing isotonic solution of dextrose.

In a preferred embodiment, the compounds of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving complex are also suitable for orally administered compositions of the invention. In these later platforms, fluid from the environment surrounding the capsule is imbibed by the driving complex, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of a compound of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the compositions can be delivered in single phase, bi-phasic, or tri-phasic systems in order to deliver the composition. Delivery of the aerosol includes the necessary container, activators, valves, subcontainers, Spacers and the like, which together can form a kit. Preferred aerosols can be determined by one skilled in the art, without undue experimentation.

Whether in solid, liquid or gaseous form, the compositions of the present invention can comprise an additional therapeutically active agent selected from among those including, but not limited to, an additional anticancer agent, an antiemetic agent, a hematopoietic colony stimulating factor, an anti-depressant and an analgesic agent.

The pharmaceutical compositions can be prepared using methodology well known in the pharmaceutical art. For example, a composition intended to be administered by injection can be prepared by combining a compound of the invention with water to form a solution. A surfactant can be added to facilitate the formation of a homogeneous solution or suspension. Surfactants are complexes that can non-covalently interact with a compound of the invention to facilitate dissolution or homogeneous suspension of the compound of the invention in the aqueous delivery system.

In one embodiment, the pharmaceutical compositions of the present invention may comprise one or more known therapeutically active agents.

In another embodiment, the pharmaceutical compositions of the present invention can be administered prior to, at the same time as, or after an additional anticancer agent, or on the same day, or within 1 hour, 2 hours, 12 hours, 24 hours, 48 hours, 72 hours, 1 week, 2 weeks, 3 weeks or 4 weeks of each other.

In another embodiment, the pharmaceutical compositions of the present invention can be administered prior to, at the same time as, or after an antiemetic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In another embodiment, the pharmaceutical compositions of the present invention can be administered prior to, at the same time as, or after an opioid or non-opioid analgesic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after an anti-depressant agent, or on the same day, or within 1 hour, 2 hours, 12 hours, 24 hours, 48 hours or 72 hours of each other.

### Kits

The invention encompasses kits that can simplify the administration of a compound or composition of the invention to a subject.

A typical kit of the invention comprises a unit dosage of a compound of the invention. In one embodiment, the unit dosage form is in a container, which can be sterile, containing an effective amount of a compound of the invention and a pharmaceutically acceptable carrier or vehicle. In another embodiment, the unit dosage form is in a container containing an effective amount of a compound of the invention as a lyophilate. In this instance, the kit can further comprise a second container which contains a solution useful for the reconstitution of the lyophilate, such as saline or phosphate buffered saline. The kit can also comprise a label or printed instructions for use of a compound of the invention. The kit can further comprise a unit dosage form of another therapeutically active agent. In one embodiment, the kit comprises a container containing an amount of an additional anticancer agent effective to treat cancer. In another embodiment the kit comprises a container containing a therapeutically active agent such as an antiemetic agent, a hematopoietic colony-stimulating factor, an analgesic agent or an anxiolytic agent.

In one embodiment, the kit comprises a unit dosage form of a pharmaceutical composition of the invention.

Kits of the invention can further comprise a device that is useful for administering the unit dosage forms of a compound or pharmaceutical composition of the invention. Examples of such devices include, but are not limited to, a syringe, a drip bag, a patch or an enema, which optionally contain the unit dosage forms.

### Therapeutic Uses Treatment of Cancer

Cancer or a neoplastic disease, including, but not limited to, neoplasms, tumors, metastases, or any disease or disorder characterized by uncontrolled cell growth, can be treated or prevented by administration of an amount of a compound of the invention effective to treat cancer or by administration of an amount of a composition effective to treat cancer, said composition comprising a pharmaceutically acceptable carrier and a compound of the invention. When the compound of the invention is a peptide platinum complex, the compositions can comprise a pharmaceutically acceptable salt thereof. The term “treating” as it applies to cancer encompasses both the treatment and/or prevention of cancer or neoplastic disease as described herein.

### Animal Models of Cancer

Animal models of cancer are used to test PT complexes according to the invention for an in vivo effect on cancer. Mouse models are useful for such in vivo testing and include the following mouse models, without limitation.
Adams J M, Harris A W, Pinkert C A, Corcoran L M, Alexander W S, Cory S, Palmiter R D, Brinster R L. 1985. The c-myc oncogene driven by immunoglobulin enhancers induces lymphoid malignancy in transgenic mice. Nature 318: 533-538.
Bishop J M. 1985a. Exploring carcinogenesis with retroviral and cellular oncogenes. Progress Med Virol 32: 5-14.
Bishop J M. 1985b. Viral oncogenes. Cell 42: 23-38.
Bishop J M. 1985c. Viruses, genes, and cancer. II. Retroviruses and cancer genes. Cancer 55: 2329-2333.
Bradley A, Evans M, Kaufman M H, Robertson E. 1984. Formation of germ-line chimaeras from embryo-derived teratocarcinoma cell lines. Nature 309: 255-256.
Brown D, Kogan S, Lagasse E, Weissman I, Alcalay M, Pelicci P G, Atwater S, Bishop J M. 1997. A PMLRARcx transgene initiates murine acute pro-myelocytic leukemia. Proc Natl Acad Sci 94: 2551-2556.
Capecchi M R. 1989. The new mouse genetics: Altering the genome by gene targeting. Trends Genetics 5: 70-76.
Casanovas O, Hicklin D J, Bergers G, Hanahan D. 2005. Drug resistance by evasion of antiangiogenic targeting of VEGF signaling in late-stage pancreatic islet tumors. Cancer Cell 8: 299-309.
Castle W E, Little C C. 1909. The peculiar inheritance of pink eyes among colored mice. Science 30: 313-314.
Castle W E, Little C C. 1910. On a modified Mendelian ratio among yellow mice. Science 32: 868-870.
Chen Z, Cheng K, Walton Z, Wang Y, Ebi H, Shimamura T, Liu Y, Tupper T, Ouyang J, Li J, et al. 2012. A murine lung cancer co-clinical trial identifies genetic modifiers of therapeutic response. Nature 483: 613-617.
Costantini F, Lacy E. 1981. Introduction of a rabbit -globin gene into the mouse germ line. Nature 294: 92-94.
de The H, Lavau C, Marchio A, Chomienne C, Degos L, Dejean A. 1991. The PML-RARcx fusion mRNA generated by the t(15;17) translocation in acute promyelocytic leukemia encodes a functionally altered RAR. Cell 66: 675-684.
Doetschman T, Gregg R G, Maeda N, Hooper M L, Melton D W, Thompson S, Smithies O. 1987. Targetted correction of a mutant HPRT gene in mouse embryonic stem cells. Nature 330: 576-578.
Donehower L A, Harvey M, Slagle B L, McArthur M J, Montgomery C A Jr, Butel J S, Bradley A. 1992. Mice deficient for p53 are developmentally normal but susceptible to spontaneous tumours. Nature 356: 215-221.
Evans M J, Kaufman M H. 1981. Establishment in culture of pluripotential cells from mouse embryos. Nature 292: 154-156.
Floc'h N, Kinkade C W, Kobayashi T, Aytes A, Lefebvre C, Mitrofanova A, Cardiff R D, Califano A, Shen M M, Abate-Shen C. 2012. Dual targeting of the Akt/mTOR signaling pathway inhibits castration-resistant prostate cancer in a genetically engineered mouse model. Cancer Res 72: 4483-4493.
Goddard A D, Borrow J, Freemont P S, Solomon E. 1991. Characterization of a zinc finger gene disrupted by the t(15;17) in acute promyelocytic leukemia. Science 254: 1371-1374.
Gordon J W, Ruddle F H. 1981. Integration and stable germ line transmission of genes injected into mouse pronuclei. Science 214: 1244-1246.
Grisolano J L, Wesselschmidt R L, Pelicci P G, Ley T J. 1997. Altered myeloid development and acute leukemia in transgenic mice expressing PML-RARcx under control of cathepsin G regulatory sequences. Blood 89: 376-387.
He L Z, Tribioli C, Rivi R, Peruzzi D, Pelicci P G, Soares V, Cattoretti G, Pandolfi P P. 1997. Acute leukemia with promyelocytic features in PML/RARcx transgenic mice. Proc Natl Acad Sci 94: 5302-5307.
He L Z, Guidez F, Tribioli C, Peruzzi D, Ruthardt M, Zelent A, Pandolfi P P. 1998. Distinct interactions of PML-RARcx and PLZF-RARcx with co-repressors determine differential responses to RA in APL. Nat Genetics 18: 126-135.
He L Z, Bhaumik M, Tribioli C, Rego E M, Ivins S, Zelent A, Pandolfi P P. 2000. Two critical hits for promyelocytic leukemia. Mol Cell 6: 1131— 1141.
He L Z, Tolentino T, Grayson P, Zhong S, Warrell R P Jr, Rifkind R A, Marks P A, Richon V M, Pandolfi P P. 2001. Histone deacetylase inhibitors induce remission in transgenic models of therapy-resistant acute pro-myelocytic leukemia. J Clin Invest 108: 1321-1330.
Jacks T, Fazeli A, Schmitt E M, Bronson R T, Goodell M A, Weinberg R A. 1992. Effects of an Rb mutation in the mouse. Nature 359: 295-300.
Jaenisch R. 1976. Germ line integration and Mendelian transmission of the exogenous Moloney leukemia virus. Proc Natl Acad Sci 73: 1260-1264.
Jaenisch R, Mintz B. 1974. Simian virus 40 DNA sequences in DNA of healthy adult mice derived from preimplantation blastocysts injected with viral DNA. Proc Natl Acad Sci 71: 1250-1254.
Juvekar A, Burga L N, Hu H, Lunsford E P, Ibrahim Y H, Balmana J, Rajen-dran A, Papa A, Spencer K, Lyssiotis C A, et al. 2012. Combining a PI3K inhibitor with a PARP inhibitor provides an effective therapy for BRCAl-related breast cancer. Cancer Discovery 2: 1048-1063.
Kakizuka A, Miller W H Jr, Umesono K, Warrell R P Jr, Frankel S R, Murty V V, Dmitrovsky E, Evans R M. 1991. Chromosomal translocation t(15;17) in human acute promyelocytic leukemia fuses RARcx with a novel putative transcription factor, PML. Cell 66: 663-674.
Kinkade C W, Castillo-Martin M, Puzio-Kuter A, Yan J, Foster T H, Gao H, Sun Y, Ouyang X, Gerald W L, Cordon-Cardo C, et al. 2008. Targeting AKT/mTOR and ERK MAPK signaling inhibits hormone-refractory prostate cancer in a preclinical mouse model. J Clin Invest 118: 3051— 3064.
Lallemand-Breitenbach V, Guillemin M C, Janin A, Daniel M T, Degos L, Kogan S C, Bishop J M, de The H. 1999. Retinoic acid and arsenic synergize to eradicate leukemic cells in a mouse model of acute promye-locytic leukemia. J Exp Med 189: 1043-1052.
Lathrop A E, Loeb L. 1915a. Further investigations on the origin of tumors in mice: I. Tumor incidence and tumor age in various strains of mice. J Exp Med 22: 646-673.
Lathrop A E, Loeb L. 1915b. Further investigations on the origin of tumors in mice: II. Tumor incidence and tumor age in hybrids. J Exp Med 22: 713-731.
Lathrop A E, Loeb L. 1918. Further investigations on the origin of tumors in mice: V. The tumor rate in hybrid strains. J Exp Med 28: 475-500.
Little C C. 1911. The “Dilute” forms of yellow mice. Science 33: 896-897.
Little C C. 1913. “Yellow” and “Agouti” factors in mice. Science 38: 205.
Little C C. 1914. A possible Mendelian explanation for a type of inheritance apparently non-Mendelian in nature. Science 40: 904-906.
Lo Coco F, Avvisati G, Diverio D, Biondi A, Pandolfi P P, Alcalay M, De Rossi G, Petti M C, Cantu-Rajnoldi A, Pasqualetti D, et al. 1991. Rearrangements of the RAR-cx gene in acute promyelocytic leukaemia: Correlations with morphology and immunophenotype. Br J Haematol 78: 494-499.
Longo L, Pandolfi P P, Biondi A, Rambaldi A, Mencarelli A, Lo Coco F, Diverio D, Pegoraro L, Avanzi G, Tabilio A, et al. 1990. Rearrangements and aberrant expression of the retinoic acid receptor cx gene in acute promyelocytic leukemias. J Exp Med 172: 1571-1575.
Lowe S W, Ruley H E, Jacks T, Housman D E. 1993a. p53-dependent apoptosis modulates the cytotoxicity of anticancer agents. Cell 74: 957-967.Downloaded from http://cshprotocols.cshlp.org/ on Feb. 18,2020—Published by Cold Spring Harbor Laboratory Press
Of Model Pets and Cancer ModelsLowe S W, Schmitt E M, Smith S W, Osborne B A, Jacks T. 1993b. p53 is required for radiation-induced apoptosis in mouse thymocytes. Nature 362: 847-849.
Lunardi A, Ala U, Epping M T, Salmena L, Webster K A, Wang G, Mazzuc-chelli R, Stack E, Lis R, Patnaik A, et al. 2013. A co-clinical approach identifies mechanisms and potential therapies for androgen deprivation resistance in prostate cancer. Nat Genet 45: 747-755.
Martin G R. 1981. Isolation of a pluripotent cell line from early mouse embryos cultured in medium conditioned by teratocarcinoma stem cells. Proc Natl Acad Sci 78: 7634-7638.
Martin A, Odajima J, Hunt S L, Dubus P, Ortega S, Malumbres M, Barbacid M. 2005. Cdk2 is dispensable for cell cycle inhibition and tumor suppression mediated by p27^{Kip1} and p21^{Cip1}. Cancer Cell 7: 591-598.
Mellinghoff I K, Wang M Y, Vivanco I, Haas-Kogan D A, Zhu S, Dia E Q, Lu K V, Yoshimoto K, Huang J H, Chute D J, et al. 2005. Molecular determinants of the response of glioblastomas to EGFR kinase inhibitors. N Engl J Med 353: 2012-2024.
Nadeau J H. 2002. Single-nucleotide polymorphisms: Tackling complexity. Nature 420: 517-518.
Nardella C, Lunardi A, Patnaik A, Cantley L C, Pandolfi P P. 2011. The APL paradigm and the “co-clinical trial” project. CancerDiscovery 1:108-116.
Nasr R, Guillemin M C, Ferhi O, Soilihi H, Peres L, Berthier C, Rousselot P, Robledo-Sarmiento M, Lallemand-Breitenbach V, Gourmel B, et al. 2008. Eradication of acute promyelocytic leukemia-initiating cells through PML-RARA degradation. Nat Med 14: 1333-1342.
Palmiter R D, Brinster R L. 1985. Transgenic mice. Cell 41: 343-345.
Pandolfi P P. 1996. PML, PLZF and NPM genes in the molecular pathogenesis of acute promyelocytic leukemia. Haematologica 81: 472-482.
Pandolfi P P, Grignani F, Alcalay M, Mencarelli A, Biondi A, LoCoco F, Grignani F, Pelicci P G. 1991. Structure and origin of the acute promyelocytic leukemia myl/RARα cDNA and characterization of its retinoid-binding andtransactivation properties. Oncogene6: 1285-1292.
Politi K, Pao W. 2011. How genetically engineered mouse tumor models provide insights into human cancers. J Clin Oncol 29: 2273-2281.
Rego E M, He L Z, Warrell R P Jr, Wang Z G, Pandolfi P P. 2000. Retinoic acid (RA) and As2O3 treatment in transgenic models of acute promyelocytic leukemia (APL) unravel the distinct nature of the leukemogenic process induced by the PML-RARα and PLZF-RARα oncoproteins. Proc Natl Acad Sci 97: 10173-10178.
Ruddle F H. 1981. A new era in mammalian gene mapping: Somatic cell genetics and recombinant DNA methodologies. Nature 294: 115-120.
Sharpless N E, Depinho R A. 2006. The mighty mouse: Genetically engineered mouse models in cancer drug development. Nat Rev Drug Discov 5: 741-754.
Stewart T A, Pattengale P K, Leder P. 1984. Spontaneous mammary adeno-carcinomas in transgenic mice that carry and express MTV/myc fusion genes. Cell 38: 627-637.
Thomas K R, Capecchi M R. 1987. Site-directed mutagenesis by gene targeting in mouse embryo-derived stem cells. Cell 51: 503-512.
Toogood P L, Harvey P J, Repine J T, Sheehan D J, VanderWel S N, Zhou H, Keller P R, McNamara D J, Sherry D, Zhu T, et al. 2005. Discovery of a potent and selective inhibitor of cyclin-dependent kinase 4/6. J Med Chem 48: 2388-2406.
Warrell R P Jr, He L Z, Richon V, Calleja E, Pandolfi P P. 1998. Therapeutic targeting of transcription in acute promyelocytic leukemia by use of an inhibitor of histone deacetylase. J Natl Cancer Inst 90: 1621— 1625.

### Therapeutic Methods

In a preferred embodiment, the present invention provides methods for treating cancer, including: killing a cancer cell or neoplastic cell; inhibiting the growth of a cancer cell or neoplastic cell; inhibiting the replication of a cancer cell or neoplastic cell; or ameliorating a symptom thereof, said methods comprising administering to a subject in need thereof an amount of a compound of the invention effective to treat cancer.

The compounds of the invention can be used accordingly in a variety of settings for the treatment of various cancers. Without being bound by theory, in one embodiment, a peptide platinum complex of the invention can enter a cell by diffusion and react with DNA to form inter-strand and intra-strand cross-links and DNA-protein crosslinks, which can interfere with the ability of the cell to replicate.

In a specific embodiment, the subject in need of treatment has previously undergone treatment for cancer. Such previous treatments include, but are not limited to, prior chemotherapy, radiation therapy, surgery or immunotherapy, such as cancer vaccines.

In another embodiment, the cancer being treated is a cancer which has demonstrated sensitivity to platinum therapy or is known to be responsive to platinum therapy. Such cancers include, but are not limited to, small-cell lung cancer, non-small cell lung cancer, ovarian cancer, breast cancer, bladder cancer, testicular cancer, head and neck cancer, colorectal cancer, Hodgkin's disease, leukemia, osteogenic sarcoma, and melanoma.

In still another embodiment, the cancer being treated is a cancer which has demonstrated resistance to platinum therapy or is known to be refractory to platinum therapy. Such refractory cancers include, but are not limited to, cancers of the cervix, prostate, and esophagus. A cancer may be determined to be refractory to a therapy when at least some significant portion of the cancer cells are not killed, or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced or has increased. Such cancers include, but are not limited to, cancers of the cervix, prostate, and esophagus.

Other cancers that can be treated with the compounds of the invention include, but are not limited to, cancers disclosed below in Table 3 and metastases thereof.

TABLE 3

| | |
|---|---|
| Solid tumors, including but not | fibrosarcoma |
| limited to: | myxosarcoma |
| | liposarcoma |
| | chondrosarcoma |
| | osteogenic sarcoma |
| | chordoma |
| | angiosarcoma |
| | endotheliosarcoma |
| | lymphangiosarcoma |
| | lymphangioendotheliosarcoma |
| | synovioma |
| | mesothelioma |
| | Ewing's tumor |
| | leiomyosarcoma |
| | rhabdomyosarcoma |
| | colon cancer |
| | colorectal cancer |
| | kidney cancer |
| | pancreatic cancer |
| | bone cancer |
| | breast cancer |
| | ovarian cancer |
| | prostate cancer |
| | esophageal cancer |
| | stomach cancer |
| | oral cancer |
| | nasal cancer |
| | throat cancer |
| | squamous cell carcinoma |
| | basal cell carcinoma |
| | adenocarcinoma |
| | sweat gland carcinoma |
| | sebaceous gland carcinoma |
| | papillary carcinoma |
| | papillary adenocarcinomas |
| | cystadenocarcinoma |
| | medullary carcinoma |
| | bronchogenic carcinoma |
| | renal cell carcinoma |
| | hepatoma |
| | bile duct carcinoma |
| | choriocarcinoma |
| | seminoma |
| | embryonal carcinoma |
| | Wilms' tumor |
| | cervical cancer |
| | uterine cancer |
| | testicular cancer |
| | small cell lung carcinoma |
| | bladder carcinoma |
| | lung cancer |
| | epithelial carcinoma |
| | glioma |
| | glioblastoma multiforme |
| | astrocytoma |
| | medulloblastoma |
| | craniopharyngioma |
| | ependymoma |
| | pinealoma |
| | hemangioblastoma |
| | acoustic neuroma |
| | oligodendroglioma |
| | meningioma |
| | skin cancer |
| | melanoma |
| | neuroblastoma |
| | Retinoblastoma |
| blood-borne cancers, including | acute lymphoblastic leukemia “ALL” |
| but not limited to: | acute lymphoblastic B-cell leukemia |
| | acute lymphoblastic T-cell leukemia |
| | acute myeloblastic leukemia “AML” |
| | acute promyelocytic leukemia “APL” |
| | acute monoblastic leukemia |
| | acute erythroleukemic leukemia |
| | acute megakaryoblastic leukemia |
| | acute myelomonocytic leukemia |
| | acute nonlymphocyctic leukemia |
| | acute undifferentiated leukemia |
| | chronic myelocytic leukemia “CML” |
| | chronic lymphocytic leukemia “CLL” |
| | hairy cell leukemia |
| | multiple myeloma |
| acute and chronic leukemias: | lymphoblastic |
| | myelogenous |
| | lymphocytic |
| | myelocytic leukemias |
| Lymphomas: | Hodgkin's disease |
| | non-Hodgkin's Lymphoma |
| | Multiple myeloma |
| | Waldenström's macroglobulinemia |
| | Heavy chain disease |
| | Polycythemia vera |

In one embodiment, the cancer is selected from the group consisting of pancreatic cancer, colorectal cancer, mesothelioma, a malignant pleural effusion, peritoneal carcinomatosis, peritoneal sarcomatosis, renal cell carcinoma, small cell lung cancer, non-small cell lung cancer, testicular cancer, bladder cancer, breast cancer, head and neck cancer, and ovarian cancer.

In a preferred embodiment the cancer is renal cell carcinoma, pancreatic cancer, colorectal cancer or mesothelioma.

### Prophylactic Methods

The compounds of the invention can also be administered to prevent progression to a neoplastic or malignant state, including but not limited to the cancers listed in Table 1. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.). Hyperplasia is a form of controlled cell proliferation involving an increase in cell number in a tissue or organ, without significant alteration in structure or function. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

The prophylactic use of the compounds of the invention is also indicated in some viral infections that may lead to cancer. For example, human papilloma virus can lead to cervical cancer (see, e.g., Hernandez-Avila et al., Archives of Medical Research (1997) 28:265-271), Epstein-Barr virus (EBV) can lead to lymphoma (see, e.g., Herrmann et al., J Pathol (2003) 199(2):140-5), hepatitis B or C virus can lead to liver carcinoma (see, e.g., El-Serag, J Clin Gastroenterol (2002) 35(5 Suppl 2):S72-8), human T cell leukemia virus (HTLV)-I can lead to T-cell leukemia (see e.g., Mortreux et al., Leukemia (2003) 17(1):26-38), human herpesvirus-8 infection can lead to Kaposi's sarcoma (see, e.g., Kadow et al., Curr Opin Investig Drugs (2002) 3(11):1574-9), and Human Immune deficiency Virus (HIV) infection contribute to cancer development as a consequence of immunodeficiency (see, e.g., Dal Maso et al., Lancet Oncol (2003) 4(2):110-9).

In other embodiments, a patient which exhibits one or more of the following predisposing factors for malignancy can treated by administration of an effective amount of a compound of the invention: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In another specific embodiment, a composition of the invention is administered to a human patient to prevent progression to breast, colon, ovarian, or cervical cancer.

### Multi-Modality Therapy for Cancer

The compounds of the invention can be administered to a subject that has undergone or is currently undergoing one or more additional anticancer treatment modalities including, but not limited to, surgery, radiation therapy, or immunotherapy, such as cancer vaccines.

In one embodiment, the invention provides methods for treating cancer comprising (a) administering to a subject in need thereof an amount of a compound of the invention effective to treat cancer; and (b) administering to said subject one or more additional anticancer treatment modalities including, but not limited to, surgery, radiation therapy, or immunotherapy, such as a cancer vaccine.

In one embodiment, the additional anticancer treatment modality is radiation therapy.

In another embodiment, the additional anticancer treatment modality is surgery.

In still another embodiment, the additional anticancer treatment modality is immunotherapy.

In a specific embodiment, the compound of the invention is administered concurrently with radiation therapy. In another specific embodiment, the additional anticancer treatment modality is administered prior or subsequent to administration of a compound of the invention, preferably at least an hour, five hours, 12 hours, a day, a week, a month, more preferably several months (e.g., up to three months), prior or subsequent to administration of a compound of the invention.

When the additional anticancer treatment modality is radiation therapy, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, the invention provides methods of treatment of cancer with a compound of the invention as an alternative to chemotherapy or radiation therapy where the chemotherapy or the radiation therapy has proven or can prove too toxic, e.g., results in unacceptable or unbearable side effects, for the subject being treated. The subject being treated can, optionally, be treated with another anticancer treatment modality such as surgery, radiation therapy or immunotherapy, depending on which treatment is found to be acceptable or bearable.

The compounds of the invention can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the patient's remaining bone-marrow cell population is then eradicated via the administration of a high dose of a compound of the invention with or without additional anticancer agents and/or high dose radiation therapy, and the stem cell graft is infused back into the animal. Supportive care is then provided while bone marrow function is restored and the subject recovers.

### Multi-Drug Therapy for Cancer

The present invention also provides methods for treating cancer comprising administering to a subject in need thereof an amount of a compound of the invention effective to treat cancer and one or more additional anticancer agents or pharmaceutically acceptable salts thereof, said additional anticancer agents not being compounds of the invention. The combination of agents can act additive or synergistic. Suitable additional anticancer agents include, but are not limited to, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil (5-fluorouracil), taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the additional anticancer agent can be, but is not limited to, a drug listed in Table 4.

TABLE 4

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum containing | Cisplatin |
| complexes: | Carboplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase | |
| Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase | Mycophenolic acid |
| Inhibitors: | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| Angiogenesis Inhibitors: | Angiostatin (plasminogen fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII fragment) |
| | Fibronectin fragment |
| | Gro-beta |
| | Halofuginone |
| | Heparinases |
| | Heparin hexasaccharide fragment |
| | HMV833 |
| | Human chorionic gonadotropin (hCG) |
| | IM-862 |
| | Interferon alpha/beta/gamma |
| | Interferon inducible protein (IP-10) |
| | Interleukin-12 |
| | Kringle 5 (plasminogen fragment) |
| | Marimastat |
| | Metalloproteinase inhibitors (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta (TGF-b) |
| | Vasculostatin |
| | Vasostatin (calreticulin fragment) |
| | ZD6126 |
| | ZD 6474 |
| | farnesyl transferase inhibitors (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Additional anticancer agents that can be used in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin II (including recombinant interleukin II, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anticancer drugs that can be used include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In a preferred embodiment, the additional anticancer agent is gemcitabine, capecitabine or 5-fluorouracil.

### Other Therapeutic Agents

The present methods can further comprise the administration of a compound of the invention and another therapeutically active agent or pharmaceutically acceptable salt thereof. The compound of the invention and the therapeutically active agent can act additively or, more preferably, synergistically. In a preferred embodiment, a compound of the invention is administered concurrently with the administration of one or more other therapeutically active agents, which can be part of the same composition or in a different composition from that comprising the compound of the invention. In another embodiment, a compound of the invention is administered prior to or subsequent to administration of one or more other therapeutically active agents. Kits comprising a compound of the invention, preferably purified, and one or more therapeutically active agents, in one or more containers are also provided.

In the present methods for treating cancer the other therapeutically active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

In a preferred embodiment, the antiemetic agent is granisetron or ondansetron.

In another embodiment, the other therapeutically active agent can be an hematopoietic colony stimulating factor. Suitable hematopoietic colony stimulating factors include, but are not limited to, filgrastim, sargramostim, molgramostim and epoietin alfa.

In still another embodiment, the other therapeutically active agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the other therapeutically active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

### EXAMPLES

### Example 1: Synthesis Cis-[Trans-(1R,2R)-1,2-Diamino-Cyclohexane]Diodoplatinum(H), (10)

A filtered solution of K₂PtCl₄ (12.51 g, 30.14 mmol) (Alfa Aesar, Ward Hill, MA; or equivalent)) in water (100 mL) was added to a solution of KI (29.19 g, 176 mmol) in water (500 mL) at 25° C. and stirred for 10 min. To the resultant solution was slowly added a solution of trans-(1R,2R)-1,2-diaminecyclohexane (3.64 g, 31.88 mmol) (12) (Alfa Aesar, or equivalent) in deionized water (130 mL). The reaction mixture was stirred for 3 hours at 25° C., filtered, and the resultant yellow precipitate washed with 3×200 mL of deionized water. The precipitate was suspended in 800 mL of deionized water, filtered, and the solids washed with 6×150 mL of deionized water at which point the filtrate showed no positive reaction towards AgNO₃. The solids were re-suspended in dimethylformamide (DMF) (150 mL) and filtered. The filter cake was then washed with DMF (3 mL), water (3×100 mL) and acetone (3×7 mL). The solids were collected and dried under reduced pressure to provide cis-[trans-(1R,2R)-1,2-diaminocyclohexane]diiodoplatinum(II) (10) as a light yellow powder (15.28 g, 90%).

### Example 2: Preparation of Diaminocyclohaxane-Carbonato Platinate (II) (15) [A Novel Platinum Compound]

To DACHPtCl₂ (23.602 mg; 62.08 μmol) or DACHPtI₂ (35.0 mg; 62.08 μmol) ([diaminocyclohexane]diiodoplatinum(II)) in Water (0.5 mL), Ag₂SO₄ (19.374 mg; 62.14 μmol) was added as a solution in 0.5 mL of Water, sonicated and then stirred for 24 h. Solution was filtered to remove precipitate; to this solution was added 12.83 mg (65.01 μg) of BaCO₃ and stirred at room temperature for 24 hours.

The resulting mixture was filtered to remove the precipitated BaSO₄ and the filtrate containing diaminocyclohaxane carbonato platinate (II) (15) (chemical structure shown below) was dried in vacuo for 24 to 48 hours and then stored dry.

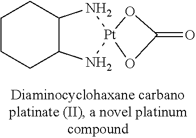

### Example 3: Peptide Synthesis: ACGPSRVGGCNH2, Silver Salt, (12)

To AcGPSRVGGCNH₂HCl (SEQ ID NO: 145) (23.602 mg; 12.176 μmol) Water (0.5 mL) was added and material was dissolved. 1N HCl solution (1 mL) was added subsequently and the solution was mixed thoroughly. After 20 min. the solution was evaporated in a stream of Nitrogen, and the resulting residue was dried in vacuo for 24 h.

To DACHPtCl₂ (23.602 mg; 62.08 μmol) in Water (0.5 mL), Ag₂SO₄ 19.374 mg (62.14 μmol) was added as a solution in 0.5 mL of Water. Sonicated and then stirred for 24 h. At the same time, suspension of Ba(OH)₂ (2.306 mg; 12.18 μmol) was added to Ac-GPSRVGG-HCl (SEQ ID NO: 149) obtained in a previous step in 0.5 mL of Water. Sonicated and then stirred for 24 h. Solutions from the previous step after filtering off the precipitate were combined, sonicated and stirred for 24 h. Precipitate was filtered off and then washed 2 times with Acetone (2×0.5mL). Crude product from Water solution (yellow solid) was crushed and dried in vacuo for 48 h. 5.085 mg of product was obtained.

### Example 4: Encapsulation of Diaminocyclohaxane Carbonato Platinate (II) (15) or Platinum (II) Peptide Conjugates into Liposomes

Add approximately 6 g of egg or soy phosphotidylcholine or other phospholipid into 60 mL of ethanol and to this add up to 5 mL of water, containing diaminocyclohaxane carbonato platinate (II) (11) or other peptide platinum complexes at ca. 20 mg/mL concentration (other concentrations would also be suitable) and then dry down. Once dry, resuspend in lipid layer in 100mL water with mixing and 3-5 cycles of 15 second sonication to aid with the resuspension. This preparation will yield relatively large size multilamellar vesicles ranging from 200 nm up to 1-2 μm. To generate smaller unilamellar lipid vesicles, the lipid vesicles containing Pt complexes can be extruded by passage through a thin needle or membrane under pressure, for small scale preparations, or else fluidized using an appropriate microfluidizer, for larger scale preparations. To make the preparation isotonic, either monosaccharides (sorbitol or mannitol) at 5% (W:W) concentration or disaccharides (trehalose, sucrose, . . . ) at 10% (W:W) can be added can be added to the platinum complexes encapsulated into liposomes.

Alternatively, add ca. 6 g of egg or soy phosphotidylcholine or other phospholipid into 100 mL of water and mix well by stirring or vortexing for 15 to 20 minutes. To this add 5 mL of a diaminocyclohaxane carbonato platinate (II) (11) or other peptide platinum complex solution at ca. 20 mg/mL concentration (other concentrations would also be suitable) and stir or vortex for at least 10 minutes. To form small lipid vesicles, process through microfluidizer at 15-20K psi by performing at least 5 passes. For isotonic preparation, either monosaccharides (sorbitol or mannitol) at 5% (W:W) concentration or disaccharides (trehalose, sucrose, . . . ) at 10% (W:W) can be added to the platinum complexes encapsulated into liposomes.

### Example 5: Encapsulation of Diaminocyclohaxane Carbonato Platinate (II) (15), Platinum Compounds and Platinum-Peptide Conjugates in PLGA Nanoparticles

Weigh 100 mg (+/−5 mg) of poly(lactic-co-glycolic acid) (PLGA) and place into a glass test tube. Add 1 ml of solvent (ethyl acetate (EtAc) or dichloromethane (DCM)) to the PLGA. Cover the top of the tube and let the polymer dissolve overnight, adding additional solvent the next day upon any evaporation. Alternatively, dissolve the polymer on the day of nanoparticle preparation, if needed, by vortexing on high speed until all polymer is completely dissolved.

Add 45 ml of 0.3% w/v Vitamin E-TPGS to a 200 ml glass beaker with a magnetic stir bar and stir. Transfer 2 ml of 0.3% w/v Vitamin E-TPGS into a glass test tube.

Add up to 50 μL of platinate drug in water (at 1-10 mg/mL) or buffer onto the PLGA solution. Ultrasonicate briefly to emulsify the drug with polymer (typically 10 sec), while keeping the mixture on ice. Obtain the test tube containing 2 mL of Vitamin E-TPGS and add to it the polymer/platinate drug emulsion, dropwise, while mixing with a vortexer; continue vortexing the solution (now an emulsion) for an additional 15-20 sec. Transfer the emulsified polymer to the ultra-sonicator and sonicate the emulsion in three 10 sec bursts with the test tube immersed in ice. Pause between each ten second sonication to allow the solution to cool before proceeding to the next sonication pulse.

Add 1-2 ml of 0.3% Vitamin E-TPGS from the stirring solution to the emulsion and then transfer the entire volume of the emulsion into the stirring 0.3% Vitamin E-TPGS solution. Allow the nanoparticles to harden and solvent to evaporate while stirring for up to three hours.

Harvest the formed nanoparticles with entrapped platinate molecule by centrifugation. Discard the supernatant, being careful not to disturb the nanoparticle pellet. Resuspend the nanoparticle pellet in 4-5 ml of an aqueous solution; if platinate loaded nanoparticles are to be lyophilized, then trehalose or other cryoprotectant may be added at weight to weight ratio of 1:2 (trehalose:polymer). Freeze solution at −60 to −80° C. and lyophilize for up to 72 hr. Store lyophilized particles frozen until use.

### Example 6: Encapsulation of Diaminocyclohaxane Carbonato Platinate (II) (15), Other Platinum Compounds and Platinum-Peptide Conjugates in Viral Particles

For encapsulation of platinum compounds, add ca. 1 mL of AAV (or other type) virus particles at ca 1×10¹⁴ vp/mL preparation onto a PD-10 desalting column, equilibrated with buffer containing 5 mM reducing agent (2-mercaptoethanol or dithiothreitol), 1 mM EDTA and 25 mM sodium bicarbonate at pH 9.0. Collect the exclusion volume, containing the partially dissociated viral particles and hold for several hours at room temperature to fully dissociate particles into the comprising viral proteins. To re-associate proteins into particles, apply the solution onto another PD10 equilibrated with 1 mM CaCl₂/100 mM NaCl and 10 mMTris pH 7.0 and collect exclusion volume. To the collected volume, add 50-100 mg od platinum compound and allow particle to reform at room temperature. Collect reformed viral particles, with entrapped platinum compound(s), by centrifugation at 20K×g and then resuspend viral pellet in desired buffer.

### Example 7: In Vitro Testing for Anticancer Activity in Human and Murine Cells

The following tests can be used to assess the in vitro anticancer activity of solutions of Pt complexes in human and murine cell lines

A solution of a Pt complex is added to human tumor cell lines (HT 29, B16, and PACA2) or murine tumor cell lines (L1210, CT26) cultured in 96-well plates. After 18 hours of incubation, the cells are pulsed with 3H thymidine and washed with phosphate-buffered saline (PBS). The amount of radioisotope incorporation is measured and used to calculate the effective concentration 50 (EC50), which is the concentration that causes a 50% decrease in cell proliferation. The test protocol for the L1210 model is described in Han et al., Cancer Chemother. Pharmacol. 39: 17-24 (1996). For example, (0165) cells are pulsed with 3H-Thy. The amount of isotope incorporation is measured and used to calculate the EC50, which is the concentration that causes a 50% decrease in cell proliferation. A Pt complex having an EC50 of <100 μM possesses sufficient activity to be considered useful according to the invention. A Pt complex which does not achieve a 50% decrease in cell proliferation at any concentration is not an anticancer compound according to the invention.

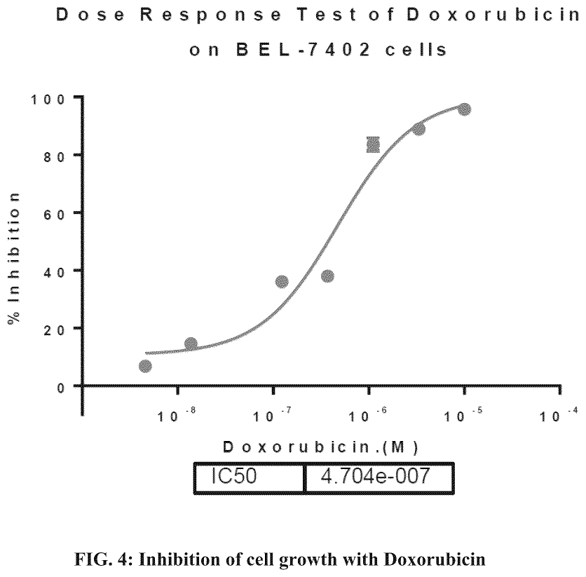

Alternatively, A375 cells (human skin melanoma derived cell line) are harvested by centrifugation and then re-suspended in a culture medium. The cell density is adjusted, and cell suspension transferred to the assay multi-well plate. The marginal wells are filled with PBS. The assay plate is incubated in a cell incubator for about 16˜18 hours. Working solutions of test compounds and positive control at various concentrations are added to corresponding wells of the plate. The assay plate is then incubated for an additional 72 hours in a 37° C./5% CO₂ incubator. After incubation, the working solution of CellTiter-Glo® reagent is added to the corresponding wells and shaken on an orbital shaker. The assay plate is incubated at room temperature for a short while to stabilize the luminescent signal. Luminescence signals are measured by a luminescence plate reader, such as PheraStar (BMG). Data are stored on a local computer or computer server for analysis. Cell killing results obtained with some of peptide platinum complexes in combination with the A375 cell line are provided in FIGS. 1, 2 and 3 for Onco-001, 003 and 005, respectively, as shown above and FIG. 4 for doxorubicin, as a control using BEL-7402 cells.

While all three Onco compounds were found to be effective at killing cells, Onco-001 was the least potent with an EC50 of ca. 130 μM; whereas, both Onco-003 and Onco-005 displayed EC50 values of less than 10 μM, indicating that Onco-003 and Onco -005 are approximately 15× more potent than Onco-001. An inactive compound is one that displays minimal or no killing activity at any concentration, even as high as at a concentration of 10-50 mM.

### Example 8: Treatment Using a Mouse Model of Cancer

The present invention also provides methods for treating cancer comprising administering to a subject in need thereof an amount of a compound of the invention effective to treat cancer and one or more additional anticancer agents or pharmaceutically acceptable salts thereof.

### Melanoma

In practice, a peptide platinum complex according to the invention is administered to a mammal, and after a sufficient time, the effect of the complex on cancer in vivo is determined according to any parameters selected by a person of ordinary skill in the art. For example, a peptide platinum complex such as Diaminocyclohaxane-Carbonato Platinate (II)-AcGPSRVGGCNH2 (SEQ ID NO: 145), whether alone or encapsulated in lipid or PGLA particles is administered intravenously or subcutaneously to two cohorts (each cohort n≥10) of xenograft transplanted mice (xenograft transplant model) for which the xenograft implant in nude mice would be human melanoma cell lines such as A-2058 or A357. Of the two cohorts, one is the control arm and the other is the treatment arm. After implant, the tumors are allowed to grow to 50 to 100 mm³, subsequently the control cohort is administered saline and the treatment cohort is administered drug at the appropriate dose. The mice are monitored for the next 3-4 weeks, monitoring and measuring tumor size. Other mice models can also be used, these models would include syngeneic transplant models such as the B16 melanoma model or genetically engineered mouse models for melanoma.

### Example 9: Treatment Using a Mouse Model of Cancer

### Breast Cancer

A similar approach to melanoma can be used for in-vivo testing of the peptide platinum complexes for breast cancer. In this case, the platinum complex would be comprised of the platinum core structure, dicyclohexylamino-platinum, complexed with the one of the breast cancer homing peptides, such as WX₁APAYQRFLX₂, (SEQ ID NO: 150), where X₁ is any amino acid, except for W and C, and X₂ is gamma-carboxyglutamic acid, and also, the N-terminal amine is acylated.

Different mouse models can be applied including xenograft implant, syngeneic transplant and genetically engineered mouse model. The experimental approach for breast cancer would be as described for the in-vivo melanoma study, except that different doses as deemed most suitable for treatment would be explored as well as routes of administration.

All references cited herein are incorporated herein by reference in their entirety and for all purposes to the same extent as if each individual publication or patent or patent application was specifically and individually indicated to be incorporated by reference ‘in its entirety for all purposes.

### Other Embodiments

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

While many methods and materials similar or equivalent to those described herein can be used in the practice or testing of the present invention, illustrative methods and materials are herein described. Other features, objects, and advantages of the invention will be apparent from the description and from the claims. In the specification and the appended claims, the singular forms also include the plural unless the context clearly dictates otherwise.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

Other embodiments are within the following claims.

1-52. (canceled)

53. A purified peptide platinum complex comprising the formula (I): 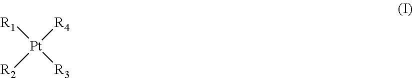
or a pharmaceutically acceptable salt thereof,
wherein
R₁ and R₂ are independently —N(R₆)₂, —NH₃⁺, or R₁ and R₂ are each —NH₂ and join through an C₂-C₆ alkylene or C₃-C₇ cycloalkylene group to form a bidentate diamine ligand;
R₃ is a peptide ligand, with the proviso that R₃ cannot be an amino acid;
R₄ is a peptide ligand, an inorganic ligand, —CN or —OC(O)R₅, with the proviso that R₄ cannot be an amino acid;
R₅ is C₁-C₂₄ alkyl; and
each R₆ is independently —H, —C₁-C₆ alkyl, —C₃-C₇ cycloalkyl or -aryl.

54. The peptide complex of claim 53, wherein said complex comprises a purified chemotherapeutic complex comprising diaminocyclohexylcarbonato-platinate (II) (11).

55. The peptide complex of claim 53, wherein said complex comprises a chemotherapeutic complex comprising diaminocyclohexylcarbonato-platinate (II) (11) encapsulated into a liposome or encapsulated into a biocompatible polymeric nanoparticle

56. The chemotherapeutic complex of claim 55, wherein said polymeric nanoparticle comprises polylacticglycolic acid (PLGA).

57. The peptide platinum complex of claim 53, where R₃ and R₄ are each, independently, a peptide and optionally wherein R₃ and R₄ are joined to form a bidentate peptide ligand.

58. The peptide platinum complex of claim 53, wherein the peptide comprises cysteine or methione.

59. The peptide platinum complex of claim 53, wherein the peptide terminal primary amino group is acylated.

60. The peptide platinum complex of claim 58, wherein the peptide comprises one or more of L-glycine, L-proline, L-serine, L-arginine, L-valine, and L-cysteine.

61. The peptide platinum complex of claim 53, wherein the peptide forms a complex with platinum via a linker.

62. The peptide platinum complex of claim 53, wherein R₁ and R₂ are joined to form a bidentate diamine ligand.

63. The peptide platinum complex of claim 61, wherein the bidentate diamine ligand is selected from the group consisting of trans-R,R-1,2-diaminocyclohexane, trans-S,S-1,2-diaminocyclohexane, cis-1,2-diaminocyclohexane and 1,2-ethylenediamine.

64. The peptide platinum complex of claim 61, wherein the bidendate diamine ligand comprises trans-R,R-1,2-diaminocyclohexane.

65. The peptide platinum complex of claim 53, wherein R₄ is an inorganic ligand, —CN or —OC(O)R₅; wherein R₅ is C₁-C₂₄ alkyl.

66. The peptide platinum complex of claim 53, wherein R₄ is Cl⁻, Br⁻, I⁻, F⁻, NO₃⁻, CN⁻, OH⁻, H₂O, HCO₃⁻ or HSO₄⁻.

67. The peptide platinum complex of claim 53, wherein said complex is selected from the group consisting of:
a) cis-bis [AcGPSRVGGCNH₂][trans-(1R,2R)-1,2-diaminocyclohexane] platinum (II), or a pharmaceutically acceptable salt thereof, said complex or salt being in purified form;
b) cis-[AcGPSRVGGCNH₂][trans-(1R,2R)-1,2-diaminocyclohexane] platinum (II), or a pharmaceutically acceptable salt thereof, said complex or salt being in purified form;
c) cis-bis [AcGPSRVGGCNH₂HCl][trans-(rac)-1,2-diaminocyclohexane] platinum (II), or a pharmaceutically acceptable salt thereof, said complex or salt being in purified form;
d) cis-[AcGPSRVGGCNH₂][trans-(rac)-1,2-diaminocyclohexane] platinum (II), or a pharmaceutically acceptable salt thereof, said complex or salt being in purified form;
e) cis-bis [AcGPSRVGGCNH₂-LINKER][cis- 1,2-diaminocyclohexane] platinum (II), or a pharmaceutically acceptable salt thereof, said complex or salt being in purified form;
f) cis-[AcGPSRVGGCNH₂-LINKER][(cis)-1,2-diaminocyclohexane] platinum (II), or a pharmaceutically acceptable salt thereof, said complex or salt being in purified form;
g) cis-bis [AcGPSRVGGCNH₂-LINKER][trans-(1R,2R)-1,2-diaminocyclohexane] platinum (II), or a pharmaceutically acceptable salt thereof, said complex or salt being in purified form;
h) cis-[AcGPSRVGGCNH₂-LINKER][trans-(1R,2R)-1,2-diaminocyclohexane] platinum (II), or a pharmaceutically acceptable salt thereof, said complex or salt being in purified form;
i) cis-bis [AcGPSRVGGCNH₂-LINKER][trans-(rac)-1,2-diaminocyclohexane] platinum (II), or a pharmaceutically acceptable salt thereof, said complex or salt being in purified form;
j) cis-[AcGPSRVGGCNH₂-LINKER][trans-(rac)-1,2-diaminocyclohexane] platinum (II), or a pharmaceutically acceptable salt thereof, said complex or salt being in purified form;
k) cis-bis [AcGPSRVGGCNH₂-LINKER][cis-1,2-diaminocyclohexane] platinum (II), or a pharmaceutically acceptable salt thereof, said complex or salt being in purified form; and
l) cis-[AcGPSRVGGCNH₂-LINKER][(cis)-1,2-diaminocyclohexane] platinum (II), or a pharmaceutically acceptable salt thereof, said complex or salt being in purified form.

68. The peptide platinum complex of claim 53, further comprising an additional anticancer agent other than the peptide platinum complex of claim 53 or a pharmaceutically acceptable salt of the peptide platinum complex of claim 53.

69. The peptide platinum complex of claim 68, wherein the additional anticancer agent comprises gemcitabine, capecitabine or 5-fluorouracil.

70. A pharmaceutical composition comprising the peptide platinum complex of claim 53 or a pharmaceutically acceptable salt of the peptide platinum complex of claim 53, in an amount effective to treat cancer, and a pharmaceutically acceptable carrier or vehicle.

71. The pharmaceutical composition of claim 70, further comprising an additional anticancer agent other than the peptide platinum complex of claim 53 or a pharmaceutically acceptable salt of the peptide platinum complex of claim 53, in an amount effective to treat cancer.

72. The pharmaceutical composition of claim 71, wherein the additional anticancer agent comprises gemcitabine, capecitabine or 5-fluorouracil.

73. A method for treating cancer, the method comprising administering to a subject in need thereof the peptide platinum complex of claim 53 or a pharmaceutically acceptable salt of the peptide platinum complex of any one of claim 53, in an amount effective to treat cancer.

74. The method of claim 73 further comprising administering to said subject an additional anticancer agent which is not the peptide platinum complex of claim 53 or the pharmaceutically acceptable salt of the peptide platinum complex of claim 53.

75. The method of claim 74 wherein the additional anticancer agent comprises gemcitabine, capecitabine or 5-fluorouracil.

76. The method of claim 75 wherein the cancer is pancreatic cancer, colorectal cancer or mesothelioma.

77. The method of claim 76, wherein the subject is a human.

78. A kit comprising a container comprising a unit dosage form of the peptide platinum complex of claim 53 or a pharmaceutically acceptable salt thereof.

79. The kit of claim 78 further comprising a second container, the second container comprising a solution for reconstitution of the peptide platinum complex.

80. The kit of claim 79, wherein the solution is an aqueous solution.

81. The kit of claim 80, wherein the aqueous solution comprises one or more of sodium chloride, phosphate buffered saline, and dextrose.

82. The kit of claim 81 wherein the aqueous solution comprising dextrose is isotonic.

83. The kit of claim 79, further comprising a third container, the third container comprising an additional anticancer agent other than the peptide platinum complex of claim 53 or a pharmaceutically acceptable salt of the peptide platinum complex of claim 53.

84. The kit of claim 83, wherein the additional anticancer agent comprises gemcitabine, capecitabine or 5-fluorouracil.

85. The kit of claim 83, further comprising a fourth container, the fourth container comprising an antiemetic agent or a hematopoietic colony stimulating factor.

86. The kit of claim 79, further comprising means for administering the peptide platinum complex of claim 53 or a pharmaceutically acceptable salt thereof to a subject.

87. A method for making a platinum complex of formula (I), 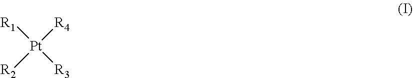
comprising allowing a complex of formula (II), 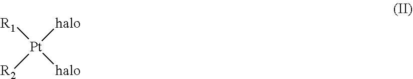
to react with at least about 2 molar equivalents of a compound of formula (II), 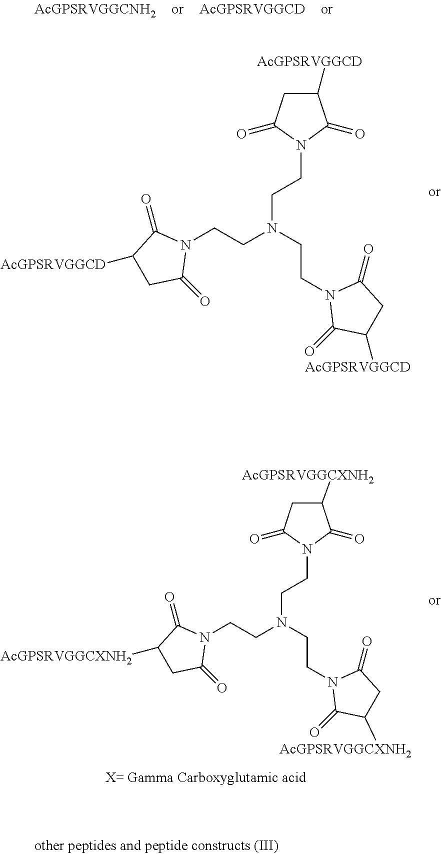
wherein
R₁ and R₂ are independently —N(R₆)₂, —NH₃⁺, or R₁ and R₂ are each —NH₂ and join through an C₂-C₆ alkylene or C₃-C₇ cycloalkylene group to form a bidentate diamine ligand;
R₃ is a peptide ligand, with the proviso that R₃ cannot be an amino acid;
R₄ is a peptide ligand, an inorganic ligand, —CN or —OC(O)R₅, with the proviso that R₄ cannot be an amino acid;
R₅ is C₁-C₂₄ alkyl;
each R₆ is independently —H, —C₁-C₆ alkyl, —C₃-C₇ cycloalkyl or -aryl; and halo is —F, —Cl, —Br, —I or —At.

88. The method of claim 87, wherein R₃ and R₄ are each independently a peptide and optionally R₁ and R₂ join to form a bidentate diamine ligand.

89. The method of claim 88, wherein the bidentate diamine ligand is trans-R,R-1,2-diaminocyclohexane, trans-S, S-1,2-diaminocyclohexane, cis-1,2-diaminocyclohexane or 1,2-ethylenediamine.

90. The method of claim 89, wherein R₃ and R₄ together comprises the peptide AcGPSRVGGCNH₂ and optionally R₃ and R₄ are attached to the same platinum moiety.

91. The method of claim 87, wherein R₃ is AcGPSRVGGCNH₂ and R₄ is an inorganic or organic group.
